(19) United States
(12) Patent Application Publication
(10) Pub. No.: US 2022/0273703 A1
(43) Pub. Date: Sep. 1, 2022

(54) NRF2 ACTIVATION FOR TREATMENT OF NEPHROGENIC DIABETES INSIPIDUS

(71) Applicant: University Of Pittsburgh-Of The Commonwealth System Of Higher Education, Pittsburgh, PA (US)

(72) Inventors: Francisco J. Schopfer, Pittsburgh, PA (US); Dario A. Vitturi Iglesias, Pittsburgh, PA (US); Soma Jobbagy, Boston, MA (US)

(73) Assignee: University Of Pittsburgh-Of The Commonwealth System Of Higher Education, Pittsburgh, PA (US)

(21) Appl. No.: 17/628,695

(22) PCT Filed: Jul. 22, 2020
(86) PCT No.: PCT/US2020/043089

§ 371 Date: Jan. 20, 2022

Related U.S. Application Data
(60) Provisional application No. 62/879,339, filed on Jul. 26, 2019.

(51) Int. Cl. A61K 33/14; A61K 45/06; A61P 7/12

(52) U.S. Cl. CPC A61K 33/14; A61K 45/06; A61P 7/12

(57) ABSTRACT

Disclosed is a method for treating or preventing nephrogenic diabetes insipidus (NDI) in a subject that includes administering to the subject a therapeutically effective amount of a Nuclear factor-erythroid 2-related factor 2 (Nrf2) inducer, thereby treating or preventing the NDI in the subject. The Nrf2 inducer may be a fumarate, a nitro fatty acid, a bardoxolone or sulforaphane. Also disclosed is a pharmaceutical composition comprising (i) a Nuclear factor-crythroid 2-related factor 2 (Nrf2) inducer and (ii) lithium or a lithium salt.

This application claims the benefit of U.S. Provisional Application No. 62/879,339, filed Jul. 26, 2019, which is incorporated herein by reference in its entirety.

### STATEMENT OF GOVERNMENT SUPPORT

This invention was made with Government support under grant number DK108391, GM125944 and DK112854 awarded by the National Institutes of Health. The government has certain rights in the invention.

### FIELD OF THE DISCLOSURE

This relates to the treatment of kidney disease, specifically to the use of a Nuclear factor-erythroid 2-related factor 2 (Nrf2) inducer to treat nephrogenic diabetes insipidus (NDI), such as lithium-induced NDI.

### BACKGROUND

Kidney disease, including chronic and acute disease, causes over 800,000 deaths worldwide each year. Acute kidney disease (AKD) involves loss of kidney function typically stemming from an acute causative event (for example, sepsis, ischemia, trauma, and/or nephrotoxic drugs). In contrast, chronic kidney disease (CKD) involves progressive loss of kidney function over a period of months or years. The pathophysiology of kidney disease varies greatly depending on the type of disease. Nephrogenic diabetes insipidus (NDI) is a kidney disease that is distinct from AKD and CKD.

For over 60 years lithium (Li) has been the gold-standard agent for prophylaxis and treatment of bipolar disorder. Its efficacy in acute treatment and chronic prevention of both manic and depressive episodes make it the first-line drug administered for long-term mood stabilization, and it remains the only therapeutic that is documented to reduce incidence of completed suicide (Song et al., Am. J. Psychiatry 174, 795-802 (2017)). Complicating its psychopharmacologic benefits, lithium exhibits a narrow therapeutic index and may cause cardiovascular, neurological, and renal sequelae (Gitlin, Int. J. Bipolar Disord. 4, 27 (2016)). Development of nephrogenic diabetes insipidus (NDI) is the most prevalent renal side effect of chronic Li administration, with over 50% of patients developing hyposthenuria and 20-40% of patients developing frank NDI with overt polyuria (Boton et al., Am. J. Kidney Dis. 10, 329-345 (1987); Grünfeld & Rossier, Nature Reviews Nephrology (2009). doi:10.1038/nrneph.2009.43). In addition to reducing quality of life, Li-induced NDI (Li-NDI) in the long term poses a more severe iatrogenic risk, as it correlates with increased progression to cCKD and ultimately renal failure (Closeet al. PLoS One 9, e90169 (2014); Kessing et al., JAMA Psychiatry 72, 1182 (2015); Aiff et al., Eur. Neuropsychopharmacol. 24, 540-544 (2014); Aiff et al. J. Psychopharmacol. 29, 608-614 (2015); Rabin EZ et al. Can. Med. Assoc. J. 121:194-8(1979); Cairns et al., Br. Med. J. (Clin. Res. Ed). (1985), doi:10.1136/bmj.290.6467.516; Garofeanu, C. G. et al., Am. J. Kid. Dis. (2005). doi:10.1053/j.ajkd.2005.01.008)

Current treatments of Li-NDI include sodium chloride cotransporter (NCC) blocking agents (e.g. thiazides) (Shirleyet et al., Clin. Sci. 63, 533-538 (1982); Walter et al., Clin. Sci. 63, 525-532 (1982); Konoshita et al., Horm. Res. 61, 63-7 (2004); Kim, et al., J. Am. Soc. Nephrol. 15, 2836-2843 (2004); Sinke, et al., Am. J. Physiol. Physiol. 306, F525-F533 (2014)) epithelial sodium channel (ENaC) inhibitors (e.g. amiloride) (Kosten & Forrest, Am. J. Psychiatry 143, 1563-1568 (1986); Kortenoeven et al., Kidney Int. 76, 44-53 (2009); Christensen et al. J. Am. Soc. Nephrol. 22, 253-261 (2011); Finch et al., Pharmacotherapy 23, 546-550 (2003); Bedford, et al. Am. J. Physiol. Physiol. 294, F812-F820 (2008); Bedfordet al., Clin. J. Am. Soc. Nephrol. 3, 1324-31 (2008); Kalita-De Croft et al., Nephrology 23, 20-30 (2018)), carbonic anhydrase (CA) inhibitors (e.g. acetazolamide) (de Groot, T. et al. Am. J. Physiol. Physiol. 313, F669-F676 (2017); de Groot, T. et al. J. Am. Soc. Nephrol. 27, 2082-2091 (2016); Gordon, et al., N. Engl. J. Med. 375, 2008-2009 (2016)), and non-steroidal anti-inflammatory drugs (NSAIDs; e.g. indomethacin) (Allen et al. Arch. Intern. Med. 149, 1123 (1989); Lam & Kjellstrand, Ren Fail 19, 183-8. (1997); Kim, et al. Am. J. Physiol. Physiol. 294, F702-F709 (2008); Kim, Electrolyte Blood Press. 6, 35-41 (2008)). The use of each of these agents to improve Li-NDI is paradoxical, as these compounds are used in other contexts to induce diuresis (thiazides, ENaC inhibitors, CA inhibitors) or are contraindicated in patients with renal disease (NSAIDs). Furthermore, the mechanisms of action of these drugs are incompletely understood, as a reduction in polyuria/polydipsia is not consistently attributable to improvement of urine osmolality and AQP2 expression. A need remains for treatments for NDI, including but not limited to Li-induced NDI.

### SUMMARY OF THE DISCLOSURE

The use of a Nrf2 inducer to treat NDI, such as lithium (Li)-induced NDI, is disclosed herein.

In one embodiment disclosed is a method for treating or preventing nephrogenic diabetes insipidus (NDI) in a subject, that includes administering to the subject a therapeutically effective amount of a Nuclear factor-erythroid-2-related factor 2 (Nrf2) inducer, thereby treating or preventing the NDI in the subject.

Also disclosed is a pharmaceutical composition comprising (i) a Nuclear factor-erythroid2-related factor 2 (Nrf2) inducer and (ii) lithium or a lithium salt.

The foregoing and other features and advantages will become more apparent from the following detailed description of several embodiments which proceeds with reference to the accompanying figures.

### BRIEF DESCRIPTION OF THE FIGURES

FIGS. 1A-1G. Lithium administration rapidly induces NDI but does not activate renal Nrf2 signaling. (A) Schematic of experimental Li-NDI model. Mice received normal chow (Control) or 0.17% dietary LiC1 (LiC1 ) for 0-7 days. (B) Water intake was significantly increased after Li administration). Results plotted as mean±standard error of 4 (Control) or 6 (LiCl) animals per group and statistical significance assessed by two-way ANOVA with Dunnet correction for multiple comparisons (C) Immunoblotting and densitometry for glycosylated ( [symbol] , 45 kDa) and non-glycosylated ( [symbol] , 29 kDa) AQP2 expression in kidney homogenates. (D) Spot urine osmolality from day 7. (E) Immunoblotting and densitometry for NADPH Quinone Dehydrogenase 1 (NQO1) protein expression in kidneys from Nrf2-/- (Nrf2 knock-out mice), mice with, Keapl floxed gene (Keap1f/f), control, and LiCl-fed mice. (F) Immunofluorescence microscopy evaluating NQO1 (green) and Mucin 1 (Mucl) (red) protein abundance with F-actin (phalloidin, white) and nuclei using 4,6-diamidino-2-phenylindole (DAPI, blue) co-stains in wild-type mouse kidney. Left: NQO1 only. Right: Merge of NQO1, Muc1, actin, and nuclei. (G) Representative immunoblotting for NQO1 in primary human renal cortical cells immunoaffinity enriched for Muc1 or CD13 and cultured in presence of 10 or 50 mM LiCl for 6 hrs. Densitometry shows average results of 3 experiments on primary human renal cortical cell lines from three separate cadaver donors and normalized to internal vehicle control; NS: not statistically significant by one-way ANOVA with Tukey correction for multiple comparisons comparing to vehicle control.

FIGS. 2A-2E. Nrf2 is not required for development of Li-NDI. (A) Schematic of experiment. (B) Animal weight, and 24hr body weight-normalized food intake over (C) and water intake (D) of Nrf2-/- mice on control diet (0-5 days) followed by LiCl diet (6-11 days). (E) Spot-urine osmolality of LiCl-treated Nrf2-/- or WT control mice at day 11.

FIGS. 3A-3M. Nrf2 hyperactivation protects against development of Li-induced nephrogenic diabetes insipidus (Li-NDI). (A) Schematic of model of Li-NDI showing groups and n per group. (B) Animal weight changes as a function of time, normalized to starting weight. 24 hr body weight-normalized food intake (C) and water intake (D). Results plotted as mean±SEM, *(p<0.05) and ***(p<0.001) denote statistical significance by two-way ANOVA with Dunnet correction for multiple comparisons, means of each time point compared to control. Plasma sodium (E), potassium (F), chloride (G), and Li+(H). (I) Urine osmolality from day 13. (J) Immunoblotting for glycosylated ( [symbol] , 45 kDa) and non-glycosylated ( [symbol] , 29 kDa) AQP2 and NQO1 expression in kidney homogenates. (K-M) Densitometry showing individual values, mean±SEM with statistical analysis by one-way ANOVA with Tukey correction for multiple comparisons.

FIGS. 4A-4G. Nrf2 hyperactivation down-regulates NCC and CA-II expression. (A) Immunoblotting of kidney lysates; each lane represents one animal from the study. (B-G) Densitometric analysis of immunoblots normalized to GAPDH showing individual values, mean±SEM. Statistical analysis by one-way ANOVA with Tukey correction for multiple comparisons.

FIG. 5. Nrf2 marker NQO1 is localized to proximal tubules in renal cortex. Immunofluorescence staining for NQO1 (green), Muc1 (red), F-actin (white) and nuclei (DAPI, blue) showing cortical predominance and proximal tubular enrichment of Nrf2 activity. DT: Distal Tubule. PT: Proximal Tubule. G: Glomerulus. V: Vessel.

FIGS. 6A-6F. Distribution of Nrf2 activity marker NQO1 and inducibility in murine kidney and cultured primary human kidney epithelial cells. (A) Immunofluorescence staining for NQO1 (green) and Mucl (red) in renal cortex and medulla reveals increased cortical co-localization and increased medullary expression in Keap1f/f mice. (B) Schematic showing distribution of constitutive and inducible Nrf2 activity in kidney. (C) Expression of NQO1 in immunoaffinity isolated primary human kidney distal tubule and cortical collecting duct cortex epithelial cells from three cadaveric donors (HAK31, HAK10, and HAK7) after CDDO-Im treatment. (D-E)

Densitometry for NQO1 (D), MUC1 (E), and CD10 (F) normalized to GAPDH expression. Results are mean +/− SEM of 3 replicates, each representing a unique human sample. Statistical testing by one-way ANOVA and significance denoted *p<0.05; ***p<0.0005.

FIGS. 7A-7J. Keap1f/f mice have endothelial dysfunction resulting in impaired vasodilation. Acetylcholine-mediated vasodilation in (A) mesenteric and (B) thoracodorsal resistance vessels. (C) EC50 of ACh in mesenteric and thoracodorsal arteries (upper panel), and vasoconstriction plot of mesenteric arteries in WT and Keap1f/f animals in response to phenylephrine. Plasma (D) and urine (E) nitrite levels as a biomarker for nitric oxide. (F) No changes in plasma arginine were detected by HPLC-MS/MS. (G) Renal expression of nNOS, eNOS, phospho-Ser1177 eNOS, sGC-B1, and NQO1 and respective densitometry normalized to GAPDH (H). (J) cGMP in plasma. (K) Mesenteric artery dilation in response to the NO donor sodium nitroprusside (SNP). Myography studies performed in technical duplicate on vessels from n=5-10 age-matched animals, relative magnitude of dilation compared by 2-way ANOVA and EC50 determined by nonlinear fit with statistical comparison by F test. Statistical analysis for densitometry and cGMP by t-test.

FIGS. 8A-8G. Constitutive Nrf2 activation suppresses cyclooxygenase-1 (COX-1) and cyclooxygenase-2 (COX-2) to down-regulate inflammation-related vasodilator production. Immunoblotting for COX-1 and COX-2 expression in kidneys of WT and Keap1f/f (A) and corresponding densitometry (B). (C) Immunohistochemistry for COX-2 in kidney sections from WT and Keap1f/f mice. (D-E) Immunoblotting and densitometry for COX-1 and COX-2 in kidneys of WT and Nrf2-/- mice. (F) Decreased levels of renal 6-keto-PGF1α, the stable metabolite of prostacyclin (PGI2) as assessed by isotope-dilution HPLC-MS/MS. (G) Relative plasma levels of kynurenine, an endothelium-derived relaxing factor formed from tryptophan metabolism, measured by HPLC-MS/MS.

FIGS. 9A-9J. Pharmacologic activation of Nrf2 using CDDO-Me protects against polydipsia in Li-NDI via an AQP2-independent mechanism. (A) Schematic of model of Li-NDI. All mice received normal chow for 0-3d and were randomized to vehicle or CDDO-Me (5 mg/kg I.P., q.d.). At day 3, mice were randomized to either normal chow or 0.17% LiC1 diet. (B) Animal weights as a function of time, normalized to starting weight. (C) 24 hr food intake normalized to body weight. (D) Changes in water intake as a % of baseline expressed as mean±SE (n=6-8 per group). *(p<0.05) denotes statistical significance compared to LiC1-Vehicle by two-way ANOVA with Dunnet correction for multiple comparisons. (E) Immunoblotting of whole-kidney lysates for glycosylated ( [symbol] , 45 kDa) and non-glycosylated ( [symbol] , 29 kDa) AQP2 and NQO1; (F-H) Densitometry showing individual values, mean±SE with statistical analysis by one-way ANOVA with Tukey correction for multiple comparisons. (I) Plasma Li concentration. (J) Spot urine osmolality at day 10. Each point represents one animal in the study. Statistical significance by two-way ANOVA with Dunnet correction for multiple comparisons.

FIG. 10. Effect of 7 consecutive days of LiCL administration through diet on weight and food intake. (A) Animal weight as a % of initial weight and (B) 24 hr body weight-normalized food intake over 7 days. (●) control chow and (o) 0.17% LiCL diet. Results plotted as mean±SEM for n=5−6, with statistical analysis by one-way ANOVA with Tukey correction for multiple comparisons.

FIGS. 11A-11N. Li induces NDI and not primary polydipsia. Mice receiving LiCl diet on baseline-clamped water intake develop significant volume depletion with hypernatremia, polycythemia. (A) Experimental design. Water and food intake were measured for 4 days for each animal; beginning at day 4, mice were randomized to control chow or LiCl chow. Mice receiving LiCl were provided their pre-determined baseline water amount daily in small sipper tube. (B) Weight (% Day 0), (C) food intake (g/day), and (D) water intake (mL/24 hr). Dark area between dashed lines shows ad libitum water intake from LiCl treated animals in FIG. 1. Plasma Na+, K+, Cl-, Hematocrit, spot urine urine osmolality (E-I). Results (B-D) plotted as mean±standard error of 6 animals per group and statistical significance assessed by two-way ANOVA with Dunnet correction for multiple comparisons. Results (E-I) plotted as mean±standard error of 6 animals per group, statistical significance assessed by t-test.

FIG. 12. Li does not induce renal NQO1 expression. Representative IF images of renal NQO1 staining in all 4 control chow and 6 LiCl diet-fed mice from FIG. 1.

FIGS. 13A-13D. Keapl hypomorphism does not induce NDI. 24-hour metabolic cage urine collection (A) shows that Keap1f/f mice are mildly polyuric at baseline. (B) Spot urine analysis reveals mild baseline hyposthenuria. 12-hour water deprivation reveals normal urine concentrating function (C) and appropriate elevation in plasma renin activity (D).

FIG. 14. Lithium treatment and hyperactivation of Nrf2 down-regulate renal expression of COX-1 and COX-2 Immunoblotting for COX-1 and COX-2 from kidneys of WT-Chow, WT-Li, and Keap1-Li mice. Semiquantitative analysis of COX-1 and COX-2 expression by densitometry.

Results showing individual values n=5-6, mean±SEM with statistical analysis by one-way ANOVA with Tukey correction for multiple comparisons.

FIG. 15. Nrf2 hyperactivation affords durable protection development of Li-induced nephrogenic diabetes insipidus (Li-NDI). 24-hour water intake in groups after 8 months of LiCl diet. Results plotted as mean of 3-day average for each animal, +SEM. N =14 WT-Ctrl (4M, 10F), 17 WT-LiCl (8M, 9F), 17 Keap1^{flox/flox} -Ctrl (12M, 5F), 13 Keap1^{flox/flox}-LiCl (9M, 4F). *** (p<0.0001) denotes statistical significance by one-way ANOVA with Tukey correction for multiple comparisons.

### DETAILED DESCRIPTION OF SEVERAL EMBODIMENTS

It is disclosed herein that a pharmaceutical composition including a Nrf2 inducer can be used to treat NDI, such as lithium (Li)-induced NDI, in a subject.

### Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

In order to facilitate review of the various embodiments of this disclosure, the following explanations of specific terms are provided:

Administration: The introduction of a composition into a subject by a chosen route. Administration can be local or systemic. For example, if the chosen route is intravenous, the composition (such as a composition including a Nrf2 inducer) is administered by introducing the composition into a vein of the subject. The term also encompasses long-term administration, such as is accomplished using a continuous release pump or a coated, implanted device.

Agent: Any substance or any combination of substances that is useful for achieving an end or result; for example, a substance or combination of substances that are Nrf2 inducers useful for treating or inhibiting NDI in a subject. Agents include proteins, peptides, nucleic acid molecules, small molecules, organic compounds, inorganic compounds, or other molecules.

Inducer or Agonist: An agent that binds to a receptor, a repressor or other biological molecule and triggers a response by that cell, such as a signaling pathway, often mimicking the action of a naturally occurring substance. An inducer can also increase a biological pathway. Nrf2 inducers activate transcription of Nrf2-dependent genes through specific binding to antioxidant response elements (ARE sequences). In some embodiments, an Nrf2 inducer can increase binding of Nrf2 to the antioxidant response element (ARE) and increase transcription of genes regulated by Nrf2 or can decrease sequestration of Nrf2 by its repressor Keap1. Nrf2 is commonly is sequestered in the cytoplasm by Keap1.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization, and so forth. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Animal: Living multi-cellular vertebrate organisms, a category that includes, for example, mammals and birds. The term mammal includes both human and non-human mammals. Similarly, the term “subject” includes both human and veterinary subjects, for example, humans, non-human primates, dogs, cats, horses, and cows.

Chronic Kidney Disease (CKD): A condition resulting in progressive loss of kidney function over a period of months or years. In several embodiments, a subject with CKD is one having a glomerular filtration rate (GFR) of less than 60 mL/min/1.73 m² for three consecutive months (see also, the National Kidney Foundation's guidelines for diagnosing CKD (Levey et al., Ann Intern. Med., 139:137-147, 2003), incorporated by reference herein in its entirety). CKD is often diagnosed in the course of screening individuals known to be at risk of CKD, such as those with high blood pressure or diabetes, or those with a family history of CKD. CKD may also be identified when it leads to one of its recognized complications, such as cardiovascular disease, anemia or pericarditis. Co-administering: The term “co-administration” or “co-administering” refers to administration of a compound disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

Control: A reference standard. In some embodiments, the control is a negative control sample obtained from a healthy patient. In other embodiments, the control is a positive control, such as a sample obtained from a patient or animal diagnosed with NDI. In still other embodiments, the control is a historical control or standard reference value or range of values (such as a previously tested control sample, such as a group of NDI patients or animals with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 68%, at least about 80%, at least about 90%, at least about 100%, at least about 150%, at least about 200%, at least about 250%, at least about 300%, at least about 350%, at least about 400%, at least about 500%, or greater than 500%.

Diabetic Nephropathy: A progressive kidney disease caused by angiopathy of capillaries in the kidney glomeruli. Diabetic nephropathy is characterized by nephrotic syndrome and diffuse glomerulosclerosis due to longstanding diabetes mellitus, and is a prime indication for dialysis. It is classified as a microvascular complication of diabetes.

These subjects generally have macroalbuminuria (urinary albumin excretion of more than 300 mg in a 24-hour collection) or macroalbuminuria and abnormal renal function as represented by an abnormality in serum creatinine, calculated creatinine clearance, or glomerular filtration rate (GFR). Clinically, diabetic nephropathy is characterized by a progressive increase in proteinuria and decline in GFR, hypertension. Subjects with diabetic nephropathy have a high risk of cardiovascular morbidity and mortality.

Inhibiting or treating a disease: Inhibiting the full development or progression of a disease or condition, for example, in a subject who is at risk for a disease, such as NDI. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology, or preventing development of a disease, such as NDI. “Prevention” is different from “treatment.”

Nephrogenic diabetes insipidus (NM): A form of diabetes insipidus characterized by the production of large quantities of dilute urine, which results from the inability of the kidney to respond to vasopressin, the primary hormone known to enable urine concentration. To avoid dehydration, those diagnosed with NDI must consume enough fluids to equal the amount of urine produced, which may be as high as 20 L, of water per day.

NDI may he congenital or acquired, with acquired NDI comprising the majority of cases. Acquired NDI is most commonly thought to stem from chronic lithium treatment, a classic treatment for bipolar disorders. This is also called “lithium-induced” NDI. Congenital NDI arises from mutations in the vasopressin receptor, V₂R, causing it to malfunction, or in the kidney water channel, resulting in a decreased ability to absorb water. Those undergoing chronic lithium treatment or possessing V₂R. mutations display no mutations in either the UT-Al or AQP2 proteins.

Symptoms of NDI include the production of large quantities of dilute urine and the subject experiencing excessive urination and excessive thirst wherein urine of the subject does not contain glucose. One test for the diagnosis of NDI includes restricting the subject from drinking water and finding that an hourly increase in osmolality of urine of the subject is less than 30 mOsm/kg per hour for at least 3 hours and the subject is not responsive to vasopressin.

Nuclear factor-erythroid 2-related factor 2 (Nri2): A transcription factor that regulates a battery of Phase II detoxification genes, genes encoding carcinogen-detoxifying enzymes and antioxidant proteins by binding to the antioxidant response element (ARE) promoter regulatory sequence. Under basal conditions, in which the redox homeostasis is maintained in cells, Nrf2 is sequestered in the cytoplasm by a protein known as Keap1, which targets Nrf2 for ubiquitination and degradation by the proteasome, and thus controls both the subcellular localization and steady-state levels of Nrf2. An exemplary Nrf2 amino acid sequence is provided in GENBANK Accession No. NP_006155.2, Apr. 15, 2002 and an exemplary mRNA encoding Nrf2 protein is provided in GENBANK Accession No. NM_006164.5, Dec. 7, 2018.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In particular embodiments suitable for administration to a subject, the carrier may be sterile, and/or suspended or otherwise contained in a unit dosage form containing one or more measured doses of the composition suitable to induce the desired response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polyuria: A dysfunction of the urinary system characterized by emission of an excessive amount of urine, normally more than 2. liters during a 24-hour period. Polyuria usually accompanies NDI.

Standard: A substance or solution of a substance of known amount, purity or concentration that is useful as a control. A standard can also be a known value or concentration of a particular substance.

Subject: Living organisms susceptible to NDI; a category that includes both human and non-human mammals, such as non-human primates.

Therapeutically effective amount: A quantity of a specified agent sufficient to achieve a desired effect in a subject being treated with that agent. For example, this can be the amount of a Nrf2 inducer useful in inhibiting and/or treating NDI. Ideally, a therapeutically effective amount of an agent is an amount sufficient to prevent, inhibit and/or treat NDI in a subject without causing a substantial cytotoxic effect in the subject. The effective amount of an agent useful for inhibiting and/or treating NDI in a subject will be dependent on the subject being treated, the severity of the disorder, and the manner of administration of the therapeutic composition.

As used herein, the singular forms “a,” “an,” and “the,” refer to both the singular as well as plural, unless the context clearly indicates otherwise. For example, the term “a polypeptide” includes single or plural polypeptides and can be considered equivalent to the phrase “at least one polypeptide.” As used herein, the term “comprises” means “includes.” Thus, “comprising a Nrf2 inducer” means “including a Nrf2 inducer” without excluding other elements. The phrase “and/or” means “and” or “or.” “About” indicates within five percent unless otherwise specified. It is further to be understood that any and all base sizes or amino acid sizes, and all molecular weight or molecular mass values, given for nucleic acids or polypeptides are approximate, and are provided for descriptive purposes, unless otherwise indicated.

Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

### Nrf2 Inducers

Illustrative Nrf2 inducers include a fumarate, a nitro fatty acid, a bardoxolone, and sulforaphane.

In certain embodiments the fumarate is a fumarate acid ester (e.g., a fumarate methyl ester, a fumarate ethyl ester, a fumarate dimethyl or diethyl ester) or fumaric acid. Illustrative fumarases include dimethyl fumarate (e.g., Tecfidera®), diroximel fumarate (Vurnerity®), tepilamide fumarate, and monomethyl fumarate.

Illustrative bardoxolones include omaveloxolone, hardoxolone methyl, and hardoxolone-imidazole (hardoxolone-Im).

Illustrative nitro fatty acids include 9-nitro-octadec-9-enoic acid, 10-nitro-octadec-9-enoic acid, 9-nitro-tetradec-9-enoic acid, 10-nitro-tetradec-9-enoic acid, 10-nitro-pentadec-10-enoic acid, 11-nitro-pentadec-10-enoic acid, 7-nitro-nonadec-7-enoic acid, 8-nitro-nonadec-7-enoic acid, 8-nitro-eicos-8-enoic acid, 9-nitro-eicos-8-enoic acid, 6-nitro-octadec-6-enoic acid, and 7-nitro-octadec-6-enoic acid.

Additional nitro fatty acids include nitro dicarboxylic acids as disclosed in PCT International Published Patent Appl. WO 2017/151938. In particular, such nitro fatty acids include those shown below.

A dicarboxylic acid having the structure:

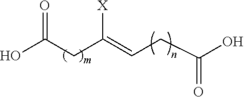

wherein X is an electron withdrawing group,
is a single or double bond,
m is from 1 to 10; and
n is from 1 to 10.

A dicarboxylic acid having the structure:

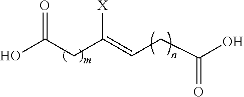

wherein X is a nitro or cyano withdrawing group,
is a single or double bond,
m is from 1 to 10; and
n is from 1 to 10.

A dicarboxylic acid having the structure:

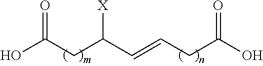

wherein X is a keto group,
is a single or double bond,
m is from 1 to 10; and
n is from 1 to 10.

A dicarboxylic acid having the structure:

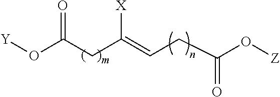

wherein X is an electron withdrawing group,
is a single or double bond,
Y and Z are each, independently, hydrogen or a C₁ to C₁₀ alkyl;
m is from 1 to 10; and
n is from 1 to 10.

A dicarboxylic acid having the structure:

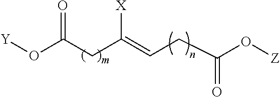

wherein X is a nitro or cyano withdrawing group,
is a single or double bond,
Y and Z are each, independently, hydrogen or a C₁ to C₁₀ alkyl;
m is from 1 to 10; and
n is from 1 to 10.

A dicarboxylic acid having the structure:

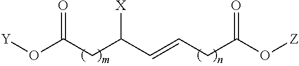

wherein X is an oxygen forming a keto group,
is a single or double bond,
Y and Z are each, independently, hydrogen or a C₁ to C₁₀ alkyl;
m is from 1 to 10; and
n is from 1 to 10.

A nitro fatty acid having a formula:

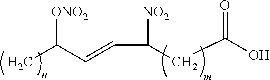

wherein n is from 1 to 10 and m is from 1 to 10.

Illustrative compounds include 15-nitro-tetracos-15-enoic acid, 16-nitro-tetracos-15-enoic acid, 14-nitro-tricos-14-enoic acid, 15-nitro-tricos-14-enoic acid, 13-nitro-docos-13-enoic acid, 14-nitro-docos-13-enoic acid, 12-nitro- heneicos-12-enoic acid, 13-nitro-heneicos-12-enoic acid, 5-nitro-eicos-5-enoic acid, 6-nitro-eicos-5-enoic acid, 9-nitro-eicos-9-enoic acid, 10-nitro-eicos-9-enoic acid, 12-nitro-eicos-11-enoic acid, 11-nitro-eicos-11-enoic acid, 8-nitro-nonadec-7-enoic acid, 10-nitro-nonadec-9-enoic acid, 9-nitro-nonadec-9-enoic acid, 8-nitro-nonadec-7-enoic acid, 12-nitro-octadec-11-enoic acid, 11-nitro-octadec-11-enoic acid, 13-nitro-octadec-13-enoic acid, 14-nitro-octadec-13-enoic acid, 10-nitro- heptadec-10-enoic acid, 11-nitro-heptadec-10-enoic acid, 10-nitro-hexadec-9-enoic acid, 9-nitro-hexadec-9-enoic acid, 8-nitro-pentadec-8-enoic acid, 9-nitro-pentadec-8-enoic acid, 7-nitro-tetradec-7-enoic acid, and 8-nitro-tetradec-7-enoic acid.

An illustrative sulforaphane is sulforaphane-cyclodextrin complex (e.g., Sulforadex).

Bardoxolone methyl and analogs thereof are disclosed for example in PCT Publication No. WO 2015/027206 and U.S. Patent Application Publication No. 2019/0091194, both incorporated by reference herein. Suitable compounds include Formula 1 (see paragraph [0013]-[0030]) and pharmaceutically acceptable salts thereof. Compound of use are also disclosed in paragraphs [0146]-[168] and include all the presented compounds on pages 18-48 of U.S. Patent Application Publication No. 2019/0091194, which is incorporated by reference in its entirety, and pharmaceutically acceptable salts thereof.

Also disclosed herein are methods of using compounds and compositions comprising acids, esters, and amides of nitro-containing fatty acids for the treatment of nephrogenic diabetes insipidus (NDI). Furthermore, the present disclosure also provides methods of using pharmaceutical compositions and oral unit dosage forms comprising acids, esters and amides of nitro-containing fatty acids for the treatment of NDI.

In one aspect, the present disclosure provides a method of treating or preventing nephrogenic diabetes insipidus (NDI) comprising administering to a subject who has NDI or is at risk of NDI an effective amount of a compound of Formula I,

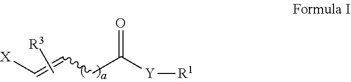

or a pharmaceutically acceptable salt, stereoisomer, and regioisomer thereof, wherein:
X is selected from H,

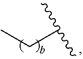

alkyl, substituted alkyl, alkenyl, nitroalkenyl, substituted alkenyl, and substituted nitroalkenyl;
Y is selected from NH, O, and S;
a is from 0-30;
b is from 0-30;
R^{l} is selected from H, alkyl, substituted alkyl, haloalkyl, substituted haloalkyl, cycloalkyl, substituted cycloalkyl, aryl, substituted aryl, heteroaryl, substituted heteroaryl, -C(O)-R², gluconate, glycoside, glucuronide, tocopherols, and PEG groups; and
R² is selected from alkyl, substituted alkyl, haloalkyl, substituted haloalkyl, cycloalkyl, substituted cycloalkyl, aryl, substituted aryl, heteroaryl, and substituted heteroaryl; and
R³ is selected from H, OH, NO₂, C(O)H, C(O)-R², COOR², COON, CN, SO₃, SO₂R², SO₃H, Cl, Br, I, F, CF₃, CHF₂, and CH₂F.
In some embodiments, the compound of Formula I is 10-nitro-9(E)-octadec-9-enoic acid according to Formula II

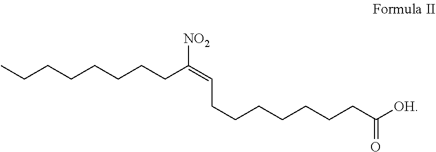 [SMILES: CCCCCCCC/C(=C\CCCCCCCC(=O)O)[N+](=O)[O-]]

In some embodiments, 10-nitro-9(E)-octadec-9-enoic acid is CXA-10.
In some embodiments, the compound of Formula I is 9-nitro-9(E)-octadec-9-enoic acid according to Formula III

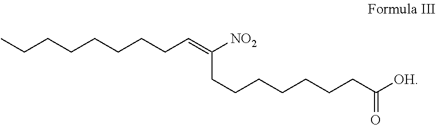 [SMILES: CCCCCCCC/C=C(\CCCCCCCC(=O)O)[N+](=O)[O-]]

In some embodiments, 9-nitro-9(E)-octadec-9-enoic acid is CXA-9.

### Methods of Treatment

Method are disclosed herein for treating or preventing NDI. These methods include administering to a subject with NDI, or at risk for developing NDI, a therapeutically effect amount of a Nrf2 inducer, as disclosed herein. The method can include selecting the subject with NDI, or at risk for developing NDI. The NDI can be acquired or can be congenital.

In some embodiments, methods are disclosed for treating or preventing congenital NDI. Congenital NDI arises from mutations in the vasopressin receptor, V₂R, causing it to malfunction, or defects in the kidney water channel, resulting in a decreased ability to reabsorb water. These methods include administering to a subject with congenital NDI, or at risk for developing congenital NDI, such as due to a genotype, a therapeutically effective amount of a Nrf2 inducer. These methods can include selecting a subject with congenital NDI, or at risk for developing congenital NDI.

In some embodiments, methods are disclosed for treating or preventing acquired NDI. Acquired NDI encompasses several clinical conditions, such as lithium-induced NDI, hypokalemic nephropathy, hypercalcemia, and post-obstructive uropathy. Any of these conditions can be treated using the methods disclosed herein. These methods include administering to a subject with acquired NDI, or at risk for developing acquired NDI, a therapeutically effective amount of a Nrf2 inducer. These methods can include selecting a subject with acquired NDI, or at risk for developing acquired NDI.

Without being bound by theory, these subjects with these conditions have low protein levels of vasopressin-regulated water channel AQP2 in the medullary collecting duct, in the presence of normal or elevated circulating levels of arginine vasopressin (A VP). In both human patients and in experimental animals with acquired NDI, the production of renal prostaglandins such as PGE2 is increased. PGE₂ has been proposed to be involved in the development of polyuria of acquired NDI.

Subject with these biological parameters can be selected for treatment.

In some embodiments, methods are disclosed for treating or preventing lithium induced NDI. These methods include administering to a subject with lithium induced NDI, or a subject taking lithium that is at risk of developing NDI a therapeutically effective amount of a Nrf2 inducer. The method can include selecting the subject.

Lithium and the salts thereof have been used to treat a variety of disorders, see PCT Publication No. 2013/033178. Lithium is approved by the U.S. FDA for the maintenance treatment of bipolar disorder and acute treatment of manic episodes of bipolar disorder. However, it is also prescribed for unlabeled uses such as treatment of neutropenia; unipolar depression; schizoaffective disorder; prophylaxis of cluster headaches; premenstrual tension; tardive dyskinesia; hyperthyroidism; postpartum affective psychosis; corticosteroid-induced psychosis. Any of these subjects can be treated using the presently disclosed methods.

Typical dosages of lithium in adult are oral administration of 900-1,800 mg/day in 2 to 4 divided doses, with a maximum daily dose of 2,400 mg/day. Pediatric dosages (in children 12. years of age and older) are 15-20 mg/kg/day administered in two to three divided doses. “Lithium” is usually administered as a lithium salt and not elemental lithium, and is typically in the form of lithium carbonate. Lithium citrate has also been used as a therapeutic agent. The pharmaceutical form of lithium has been sold under such brand names as LITHOTAB®, LITHONATE®, LITHOBID®, LITHANE®, and ESKALITH®. However, often the USAN/ΓN name of lithium carbonate or lithium citrate is used. Any subject administered these forms of lithium can he treated using the presently disclosed methods.

Acquired nephrogenic diabetes insipidus (NM) occurs in 20-50% of patients taking lithium. Without being bound by theory, lithium- induced NDI is thought to arise from an interaction of the drug with the vasopressin (AVP)-activated adenylate cyclase system in the collecting ducts of the kidney (Christensen, S., et al. (1985) J. Clin. Invest. 75: 1869-1879; Goldberg, ibid; Jackson, B. A., et al. (1980) Endocrinol. 107: 1693-1698; Yamaki, M., et al. (1.991) Am. J. Physiol. 261 :F505-F511).

The most prevalent uses of lithium are in the treatment of acute or chronic bipolar disorder and in the prevention of bipolar disorder recurrence in individuals who have experienced transient episodes. Bipolar disorder is estimated to affect approximately one percent of people throughout the world (Woods, S. W. (2000) J. Clin. Psych. 61 (suppl 13), 38-41). In the U.S., about 2% of the population affected (Lenox et al, 1998, I Clin Psychiatry 58:37—47), with double that prevalence in war veterans. Mental depression and substance abuse, which are often encountered in post-traumatic stress disorder (PTSD) patients, such as war veterans, are known to predispose them to bipolar disorder. PTSD can also result from physical or sexual abuse in childhood, physical or sexual assault in adults, serious accidents, terrorist attacks, natural disasters. A subject with any of these disorders, that also has lithium-induced NDI, can be treated using the disclosed methods. In some embodiments, the subject has bi-polar disorder and/or PTDS.

Lithium has been used to treat maladies ranging from alcoholism to unipolar depression. In the treatment of these psychological disorders, lithium is often prescribed as an augmentation of therapy when a patient is unresponsive to conventional treatment regimens. Subjects with these conditions, who also have lithium induced NDI, can be treated using the methods disclosed herein.

Methods are also disclosed for treating or preventing acquired NDI induced by other agents. The methods include administering to a subject that has the acquired NDI, or is at risk for developing the acquired NDI, a therapeutically effective amount of a Nrf2 inducer. Drugs that are capable of inducing acquired NDI include colchicine, methoxyflurane, amphotericin B, gentamicin, loop diuretics, and demeclocycline. In addition to drugs, acquired NDI can also occur as a result of certain diseases. These include, but are not limited to chronic kidney diseases, hypokalemia, hypercalcemia, sickle cell disease, ureteral obstruction (obstructive uropathy), and low protein diet. Any of these subjects can be selected for treatment.

With regard to prevention, the methods do not necessarily prevent NDI throughout the lifespan of the subject. The administration of the Nrf2 inducer to a subject at risk for NDI, such as a subject with a particular genotype or a subject that is administered lithium, can delay the development of NDI. Such a delay can be for about 1, 2, 3, 4, 5, 6, 7, 8, 9, 10, 11 or 12 months, or can be for about 1, 2, 3, 4, 5, 6, 7, 8, 9, 10 years, as compared to one or more subjects that is/are similarly at risk for developing NDI, one or more subjects with the same genotype or one or more subjects undergoing a similar treatment regimen for the same disease.

In some embodiments, using the disclosed methods, polyuria is decreased, such as by about 15%, by about 20%, by about 25%, by about 30%, by about 40%, by about 50%, by about 60%, by about 70%, by about 80%, by about 90%.

In other embodiments, using the disclosed methods, water intake is reduced in the subject, such as by about 15%, by about 20%, by about 25%, by about 30%, by about 40%, by about 50%, by about 60%, by about 70%, by about 80%, or by about 90%. In further embodiments, urine osmolality is increased in a subject with NDI, such as by about 15%, by about 20%, by about 25%, by about 30%, by about 40%, by about 50%, by about 60%, by about 70%, by about 80%, by about 90%, by about 100%, by about 150%, by about 200%, by about 250%, and by about 300%.

In some embodiments, and additional agent is administered to the subject. The additional agent can be a diuretic. Exemplary diuretics include, but are not limited to, thiazide and amiloride. The additional agent can be a P21 purinergic receptor antagonist. These antagonists include, but are not limited to, suramin, reactive blue 2, acid blue 129, acid blue 80, and pyridoxalphosphate-6-azophenyl-2′,4′-disulfonic acid (PPADS), see U.S. Patent Publication No. 20090297497A1.

In some embodiments, an additional agent is administered to the subject. The additional agent can be an NSAID. Exemplary NSAID include, but are not limited to indomethacin, acetaminophen, diclofenac, aspirin, celecoxib and ibuprofen.

In some embodiments, the Nr f2 inducer may be co-administered with lithium or a lithium salt.

Also disclosed herein is a pharmaceutical composition comprising (i) a Nuclear factor-erythroid 2-related factor 2 (Nrf2) inducer and (ii) lithium or a lithium salt.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, Pa., 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent (such as an anti-retroviral agent) for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

The disclosure is illustrated by the following non-limiting Examples.

### EXAMPLES

In murine models, Li has been demonstrated to target the epithelium lining the distal tubule (DT) and collecting duct (CD) of the nephron, where its uptake is mediated by the epithelial sodium channel (ENaC) (Kosten & Forrest, Am. J. Psychiatry 143, 1563-1568 (1986); Kortenoeven et al. Kidney Int. 76, 44-53 (2009); Christensen et al. J. Am. Soc. Nephrol. 22, 253-261 (2011)) and where it induces loss-of-function through uncoupling the effects of arginine vasopressin and down-regulating aquaporin 2 (AQP2) to yield pronounced polyuria with compensatory polydipsia (Marples et al., J. Clin. Invest. 95, 1838-1845 (1995); Kwon et al. Altered expression of renal AQPs and Na(+) transporters in rats with lithium-induced NDI. Am. J. Physiol. Renal Physiol. 279, F552-64 (2000); Thomsen & Shirley, Nephrol. Dial. Transplant. 21, 869-880 (2005)).

Nrf2 is a basic leucine zipper transcription factor which controls transcriptional responses to oxidative and electrophilic insults through upregulation of cytoprotective gene expression (Itoh et al., Biochem. Biophys. Res. Commun. 236, 313-322 (1997); Alam, J. et al., J. Biol. Chem. 274, 26071-26078 (1999); Ishii et al., J. Biol. Chem. 275, 16023-16029 (2000); Itoh et al., Free Radic. Biol. Med. 36, 1208-1213 (2004)). Under non-stressed conditions, Nrf2 is retained in the cytosol and rapidly targeted for proteasomal degradation by the Kelch like-ECH-associated protein 1 (Keap1)/Cullin3 (Cul3) complex. Redox-sensitive cysteine thiols in Keap1 sense cellular exposure to oxidative or electrophilic stress through their propensity for oxidation or alkylation, with these post-translational modifications de-repressing Nrf2 transcriptional activity (Itoh et al., Free Radic. Biol. Med. 36, 1208-1213 (2004); Kobayashi et al., Mol. Cell. Biol. 29, 493-502 (2009); Takaya et al., Free Radic. Biol. Med. 53, 817-827 (2012)).

In Chronic kidney disease (CKD) the functional parenchyma of the kidney is replaced by fibrotic nonfunctional scar tissue, leading to decrease of all functions of the kidney. The kidney filters ˜180 L of plasma per day; only ˜1-2 L of volume is excreted as urine. NDI is a distinct entity in which there is defect in the ability of the kidney to concentrate urine, leading to production of large volumes of dilute urine.

The result presented below demonstrate that activation of the Keap1/Nrf2 signaling pathway completely protects mice from polydipsia/polyuria in NDI, such as Li-NDI. The reduction in polydipsia occurs without improvement in AQP2 expression or increase in urine osmolality. Activation of Nrf2 down-regulated expression of NCC and CA-II, mimicking the effects of two common diuretic therapies for Li-NDI. Vascular effects of Nrf2 activation, and underlying reduction in inflammation-derived vasodilator production, were identified as additional contributory mechanisms. Pharmacologic activation of Nrf2 with CDDO-Me was validated as potential therapeutic intervention strategy for Li-NDI.

### Example 1

### Methods

Materials: Sodium nitrate, Ach and SNP were purchased from Sigma. Solvents were LC-MS grade from Burdick and Jackson (Muskegon, Mich.). Chemicals were of analytical grade and purchased from Sigma unless otherwise stated (St. Louis, Mo.). CDDO-Me and CDDO-Me were from Toronto Research Chemicals. Primary antibodies were purchased from the following suppliers: NQO1 (ab34173, Abcam, Cambridge, Mass.), GAPDH (Trevigen, Gaithersburg, Md.), COX-1 (Cell Signaling, Beverly, Mass.), COX-2 (ab15191 or ab179800, Abcam, Cambridge, MA), Mucin 1 (MA5-11202, Thermo), CD10 (MA5-14050, Thermo), ENaC a/0/y (StressMarq), NCC (Abcam, Cambridge, Mass.), phospho-NCC (T53; PhosphoSolutions, Aurora, CO), Carbonic Anhydrase II (Abcam, Cambridge, Mass.), Aquaporin 2 (H7661; Aarhus University, Denmark). Secondary antibodies were purchased from Santa Cruz Biotechnologies (Dallas, Tex.). Angiotensinogen 1-14, (rat sequence, DRVYIHPFHLLYYS (SEQ ID NO: 1)) and (Kortenoeven et al. Kidney Int. 76, 44-53 (2009)) C/ (Marples et al., J. Clin. Invest. 95, 1838-1845 (1995)) N labeled Angiotensin I (DR-V*-Y-I*-HPFHL (SEQ ID NO: 2)) were obtained from AnaSpec, Fremont Calif.. Zirconium oxide beads (NextAdvance, Troy N.Y.). MiniTab protease and Phos-Stop phosphatase inhibitor cocktails were from (Roche, Switzerland). Isotopically labeled standard 6-keto PGF1a-d4 was from Cayman Chemical, Ann Arbor, Mich.

Animals: Male C57BL6j/albino mice (Jackson Labs, JAX000058) at 8 weeks of age were habituated to individual housing for 4 days followed by randomization to receive control diet or diet containing 0.17% LiCl by weight (Teklad) ad libitum. Water was provided ad libitum. Mice were maintained on a 12 h light/12 h dark cycle. After 7 days, mice were sacrificed by terminal exsanguination under isoflurane anesthesia. A blood sample was collected by retroorbital bleeding, after which a laparotomy and thoracotomy were performed. A hemostat was applied to the left renal vascular bundle and the left kidney was removed and flash-frozen in liquid nitrogen. The vena cava was severed and whole-animal transcardial perfusion was performed with cold 2% paraformaldehyde (PFA) in phosphate buffered saline (PBS). The right kidney was removed, bisected, and fixed in 2% PFA for 1.5 h followed by dehydration in 30% sucrose for 24 h and freezing in temperature OCT compound. 12-hour water deprivation was performed overnight 8PM-8AM after which time animals were euthanized and tissues collected as above.

Water and Urine: Mice and food were weighed daily. Water was supplied in 50 mL conical with sipper tube as described (bio-protocol.org/e1822), and daily water intake was determined by weight. Spot urine was collected after spontaneous voiding on plastic wrap and urine osmolality measured in technical duplicate with Wescor 5500® Vapor Pressure Osmometer using lOuL urine and the average value reported each mouse.

Cell culture: Primary human distal tubule cells from human adult kidneys were isolated and cultured as previously described (Emlet et al., Am. J. Physiol.-Ren. Physiol. 312, (2017)). Confluent monolayers of cells were incubated with vehicle control, LiCl or CDDO-Im for 18 hr in serum-free hormonally defined media followed by preparation of lysates for immunoblotting.

Immunoblotting: Flash-frozen kidneys were homogenized in ice-cold Radio Immuno Precipitation Assay (RIPA) buffer containing protease and phosphatase inhibitor cocktails. Cells were treated as indicated. At time of collection, cells were washed 2× with cold PBS and lysates were prepared in 4° C. lysis buffer containing protease inhibitors. Protein was quantified by bicinchoninic acid (BCA) assay (Pierce) and sample containing 15-25 μg total cellular protein was loaded on 4-12% reducing Bis-Tris gel. Resolved proteins were transferred to 0.45 μm nitrocellulose membrane, blocked with 5% non-fat dry milk or 5% bovine serum albumin in TRIS® buffered saline (TBS)-0.1% Tween 20 for 1 hr. Membranes were incubated with primary antibody (1:1000 dilution) for 1 hr at room temperature or overnight at 4° C. Appropriate secondary antibody was applied at dilution of 1:5000 for 1 hr, membrane washed thrice in TBS-0.1% Tween-20 and membrane visualized with Clarity ECL chemiluminescence kit and ChemiDoc imager (Bio-Rad, Hercules, Calif.).

Immunofluorescence: Kidney cryostat sections (5μm) were washed three times with phosphate buffered saline (PBS), followed by 3× washes with solution of 0.5% bovine serum albumin (BSA) in PBS. Sections were blocked with 5% normal goat serum in BSA solution for 45 minutes. The slides were incubated for 1 hour at room temperature (RT) with primary antibodies for rabbit anti NQ01(ab34173, Abcam) at 1:500, and hamster anti MUC1 (MA5-11202, Thermo) at 1:50 in 0.5% BSA solution. Slides were washed three times with BSA solution and incubated for 1 hour at RT with ALEXA® 488 donkey anti mouse secondary antibody (A21202, Invitrogen) diluted 1:500, combined with goat anti rabbit CY5® (111-605-003, Jackson Immuno) 1:1000, and goat anti hamster CYS3® (127-165-160, Jackson) combined with 1:500 ALEXA® 488 phalloidin (A12379, Thermo) in BSA solution. Nuclei were stained with Hoechst dye (bisbenzamide 1 mg/100 ml water) for 30 seconds. After three rinses with PBS, sections were coverslipped with gelvatol mounting media. Images were captured with a Nikon Al confocal microscope (NIS Elements 4.4)

Determination of Plasma and Urine Nitrite: Blood was collected into tubes containing acid citrate dextrose and immediately centrifuged for 10 min at 500g to separate plasma. Plasma was aliquoted and stored −80° C. until analysis. Urine was collected onto clean plastic sheet. Nitrite levels were determined using 50 μL sample using a GE Sievers NOA 280i analyzer following manufacturer's instructions. A calibration curve containing known amounts of sodium nitrite was prepared for quantification.

Plasma Renin Concentration Assay: 30 μL plasma was added to 270 μL generation buffer (1.0M Tris 0.25M EDTA 1 mM PMSF pH 5.5) containing 30 μM Angiotensinogen 1-14 renin substrate and the reaction was incubated at 37° C. 50 μL aliquots were removed at 0, 10, 20, and 30 min and quenched with 200 μL MeOH containing 3% formic acid and (Kortenoeven et al. Kidney

Int. 76, 44-53 (2009) C/ (Marples et al., J. Clin. Invest. 95, 1838-1845 (1995)) N labeled Angiotensin I internal standard. The solution was chilled at −20° C. and protein precipitates removed by centrifugation 13,000 RPM for 10min. 20 μL of supernatant were injected for HPLC-MS/MS analysis.

Wire Myography: Endothelium-dependent and -independent relaxation responses of second order mesenteric or thoracodorsal arteries to cumulative doses of acetylcholine (Ach) and sodium nitroprusside (SNP) were evaluated using two-pin wire myography (Multiple Myograph Model 610 M, DMT; Denmark).

Determination of 6-keto prostaglandin Fla: Flash-frozen kidney samples were weighed on analytical balance and transferred to chilled Eppendorf tubes containing equivalent weight of zirconium oxide homogenization beads. 380 μL distilled water containing isotopically labeled standard 6-keto PGF1α-d4 (final concentration 50 ng/mL) was added to each sample and homogenized at 4° C. using a Bullet Blender (NextAdvance, Troy, NY). The lysate was added to 1.6 mL acetonitrile and centrifuged at 15,000 rpm for 15 min at 4° C. The supernatant was transferred to a clean glass tube and dried under N₂(g) for 1 hr. Samples were resuspended in 200 μL MeOH and 10 μL was injected for HPLC-MS/MS analysis. Analyte was quantified using 6-point calibration curve prepared for each experiment.

Determination of Plasma Amino Acids: Plasma amino acids and Kynurenine were measured using isotope-dilution HPLC-MS/MS following derivatization with phenylisothiocyanate (PITC) as previously described (Jackson et al., Cancer Res. 77, 5795-5807 (2017)).

HPLC-MS/MS: A Shimadzu HPLC (Columbia, MD) coupled to a Thermo Scientific CTC HTS PAL autosampler (Waltham, MA) and an AB Sciex (Framingham, MA) 5000 triple quadrupole mass spectrometer was used for the quantification of Angl, isotopic Angl standard, and Agt. Peptides were resolved on a Phenomenex Gemini C18 column (2.0×20 mm, 3 μm pore size) using a binary solvent system consisting of aqueous 0.1% aqueous formic acid (solvent A) and 0.1% formic acid in acetonitrile (solvent B) at a flow rate of 850 μl/min. Chromatographic conditions were as follows: 5% B for 0.3 min, followed by a linear gradient to 45% B at 2.5 min, to then move to 100% B for 1 min and re-equilibration to return to the initial condition (5% B) for 4.5 min. The triple quadrupole mass spectrometer was tuned and used in positive ion mode with the following settings: source temperature 650° C.; ionization spray voltage 5000V; CAD 5.0 arbitrary units; Curtain gas 40 arbitrary units; GS1 55 arbitrary units; GS2 55 arbitrary units; EP 10.00V; CXP 10.00V. Multiple reaction monitoring was performed with 75 ms dwell time, declustering potential 100V, collision energy 30-37V was performed using the following transitions: Ang I (Q1 433.20→Q3 110.20), isotopic Ang I (Q1 655.60→Q3 110.20), Agt (Q1 608.50→Q3 269.20). Sample Angl was calculated based on area ratio and calibration curves prepared using commercially available Angl standards, and PRC determined from slope of Angl generated by each sample as function of time.

For determination of 6-keto-PGF1α, chromatography was performed on Phenomenex Kinetex C18 column (2.1×50 mm, 5 μm pore size) using aqueous 0.1% ammonium acetate (solvent A) and 0.1% ammonium acetate in acetonitrile (solvent B) at total flow of 0.25 mL/min). 10% B was increased to 65% B over 20 min followed by 3 min wash with 100% B and return to 10% B for 2 min for a total run time of 25 min. The following settings were used in negative ion mode: Source temperature 650° C.; ionization spray voltage 5000V; CAD 5.0 arbitrary units; Curtain gas 40 arbitrary units; GS1 55 arbitrary units; GS2 55 arbitrary units; EP -5.00V; CXP -18.40V. Multiple reaction monitoring was performed with 150 ms dwell time, declustering potential −50V, collision energy -17V was performed using the following transitions: 6-keto PGF1α (Q1 369.10→Q3 245.00), 6-keto PGF1α-d4 (Q1 373.1→Q3 167.00).

### Example 2

### Dietary Administration of Li Leads to Rapid Development of NDI Without Inducing Renal Nrf2 Targets

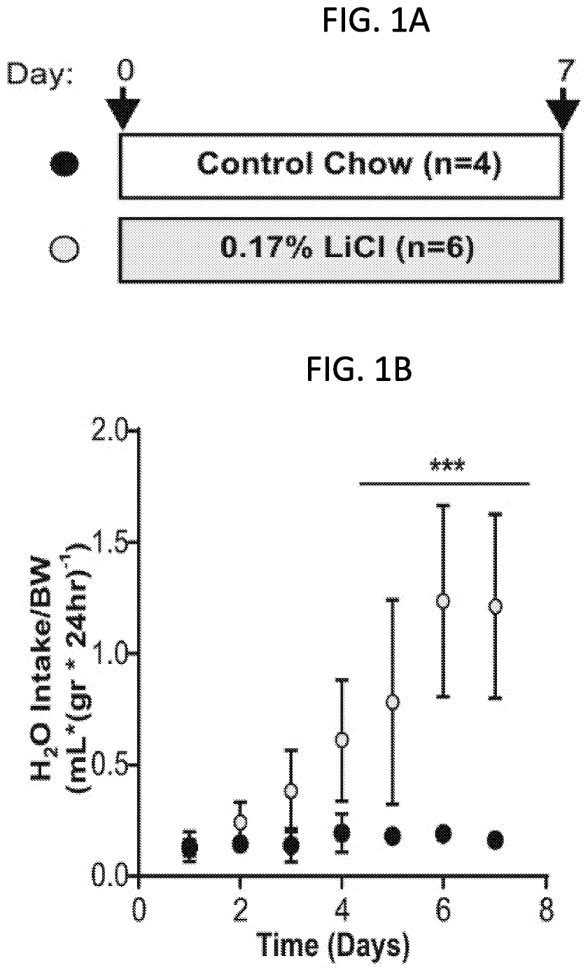

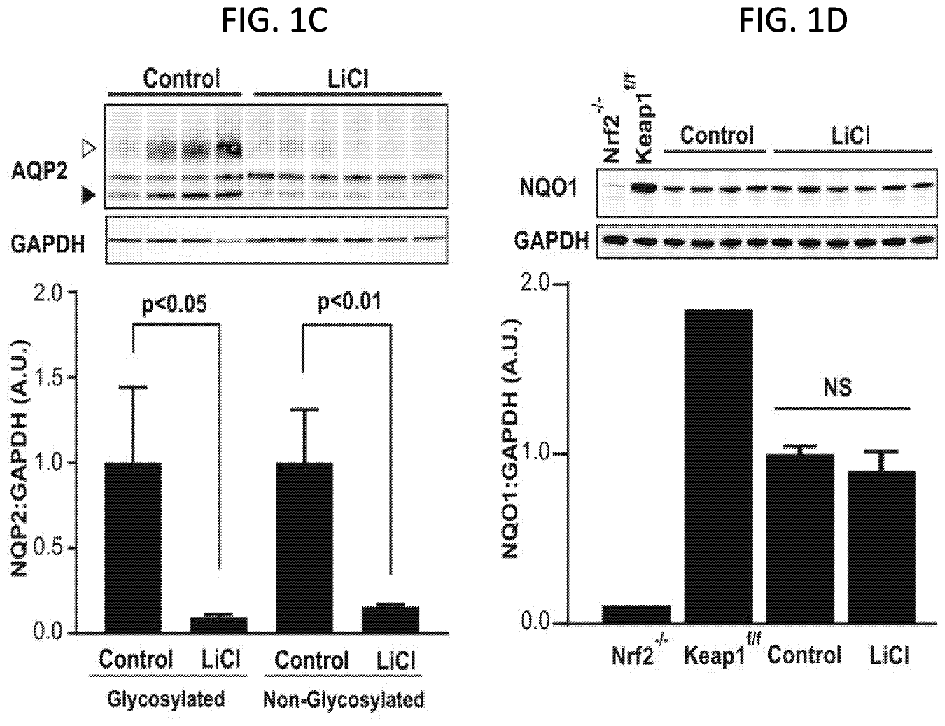

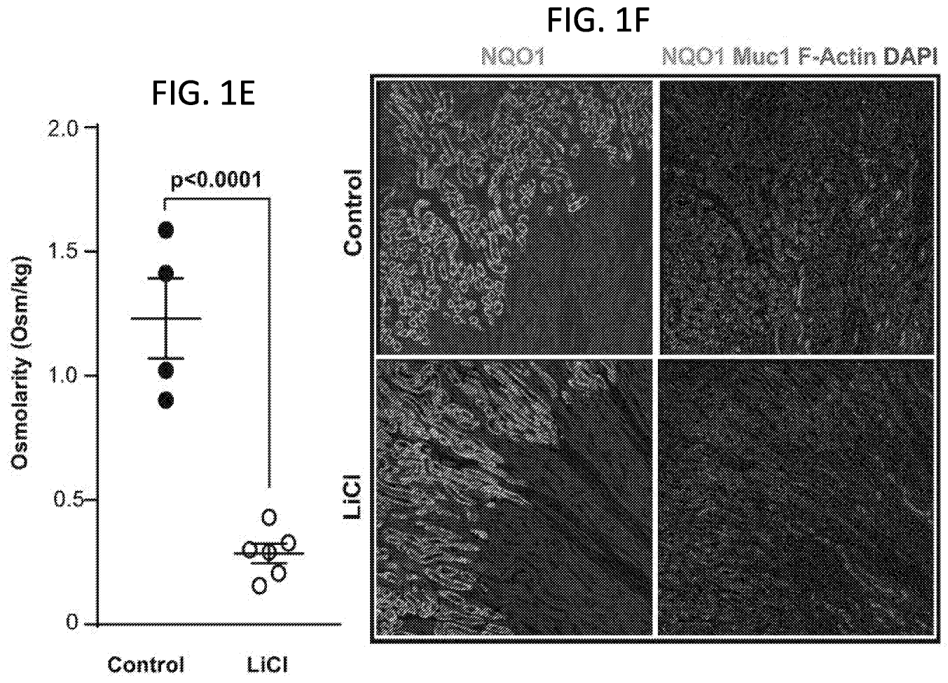

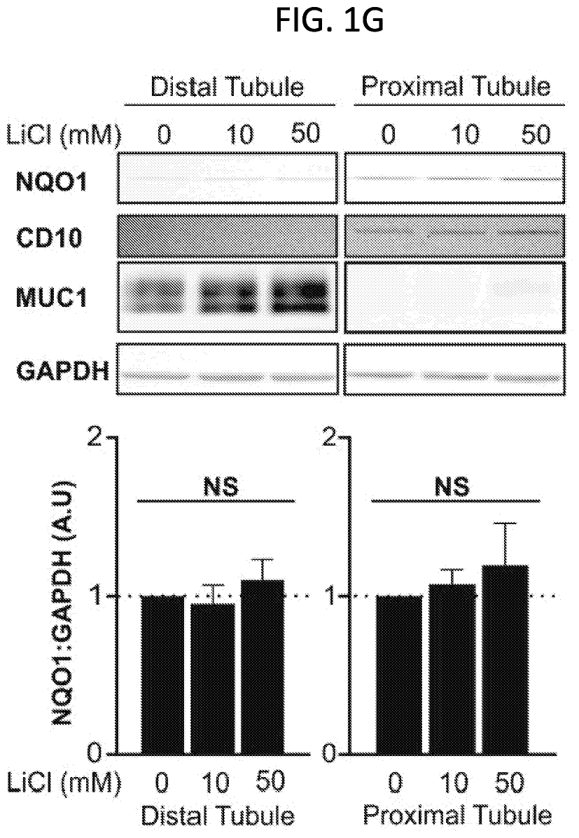

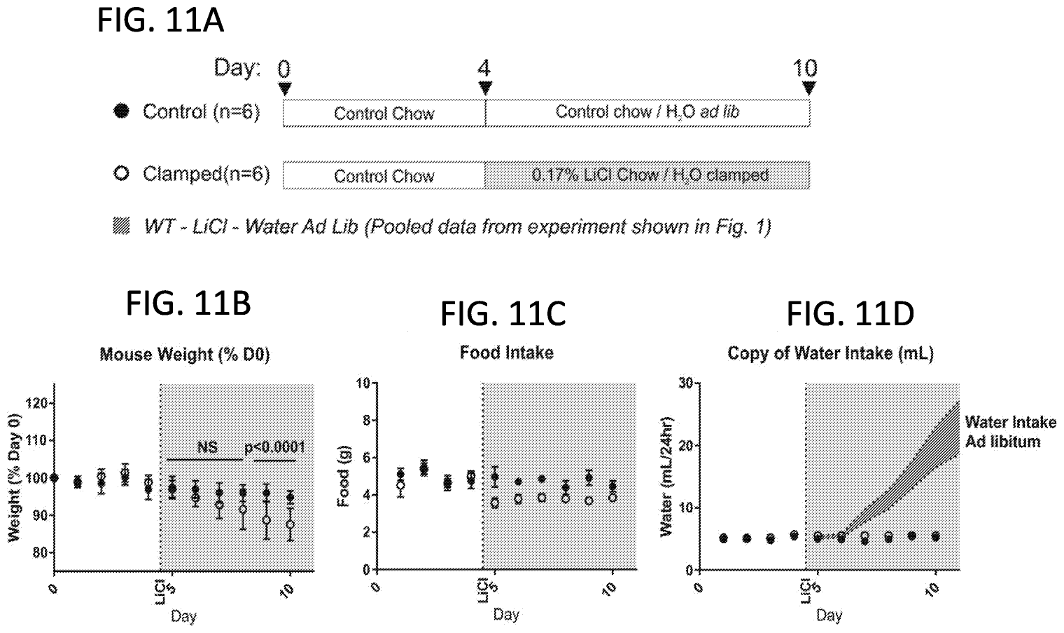

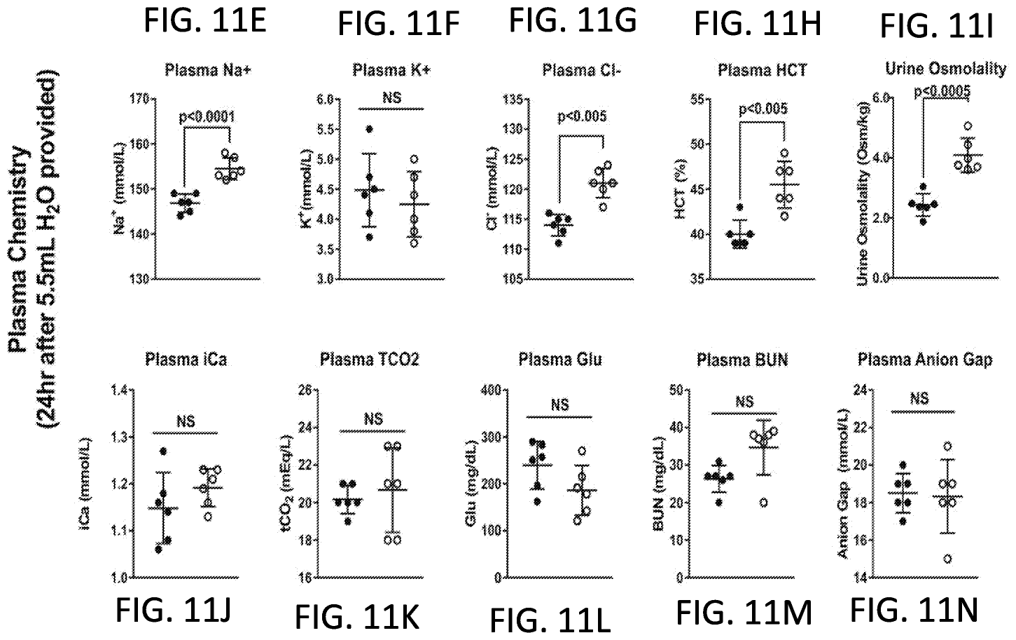

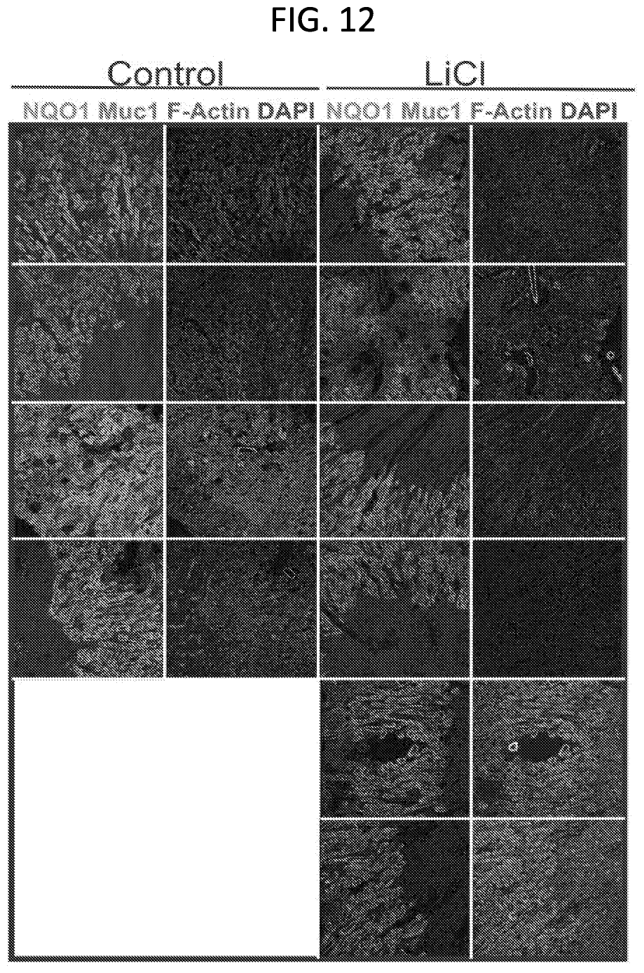

To establish a murine model of NDI, mice were administered control chow diet or 0.17% LiCl diet for 0-7 days (FIG. 1A) during which body weight, food intake (Suppl. FIG. 1) and water intake (FIG. 1B) were monitored. Water intake was used as a surrogate measurement for micturition to avoid the impact of the stress associated with metabolic cages (Kalliokoski et al., PLoS One 8, e58460 (2013), and was significantly increased in mice receiving LiCl after 4 days. By 7 days LiCl treatment significantly downregulated renal expression of AQP2 which corresponded with a significant hyposthenuria (FIG. 1 C, D), without impacting protein expression of the Nrf2 gene target NADPH Quinone Dehydrogenase 1 (NQO1) (FIG. 1E). Mice developed significant volume depletion with hypernatremia and polycythemia when water intake was clamped to match baseline intake during LiCl treatment (FIG. 11), indicating that the model recapitulated NDI and not a Li-induced primary polydipsia. While Li did not modify Nrf2 signaling activity, a large dynamic range of NQO1 expression was observed between Keap1^{f/f} (constitutive Nrf2 activity) and Nrf2 knockout (Nrf2^{-/-})(FIG. 1E). To further test for potential cell-type specific level of Nrf2 activity which could be masked in whole-tissue analysis, NQO1 expression was evaluated by immunofluorescence and likewise revealed no modulation of Nrf2 activity by Li (FIG. 1F; FIG. 12). To confirm the absence of Li-induced Nrf2 modulation, immunoaffinity isolated human cortical kidney cells enriched for distal tubule protein Mucin 1 or proximal tubule cells expressing CD10/ CD13 were incubated with Li (10 mM and 50 mM). No increase in NQO1 expression was observed on either cell type upon Li treatment (FIG. 1G).

### Example 3

### Nrf2 is Not Necessary for Development of Li-NDI in Mice

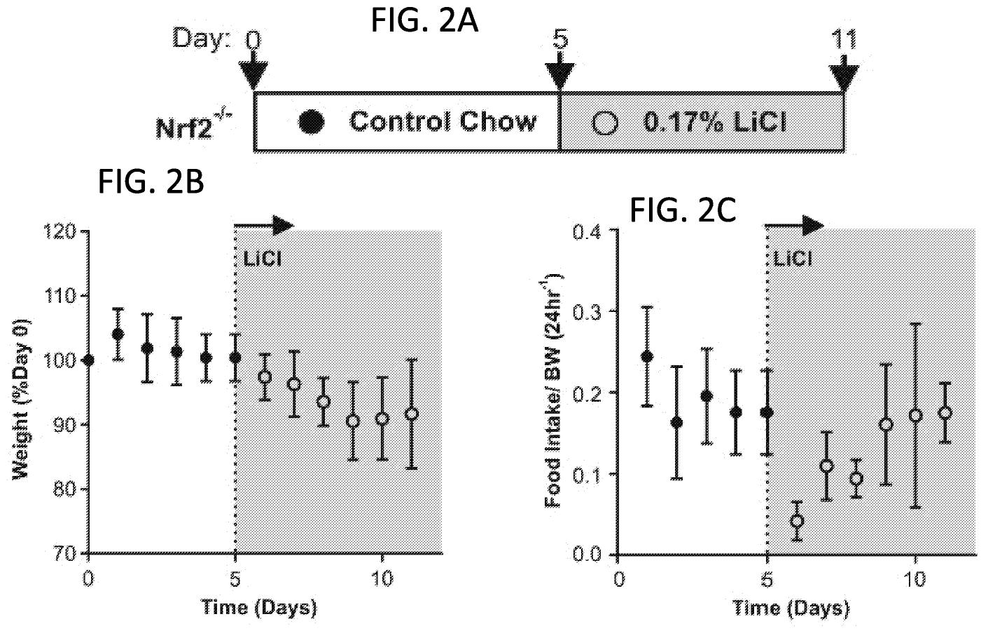

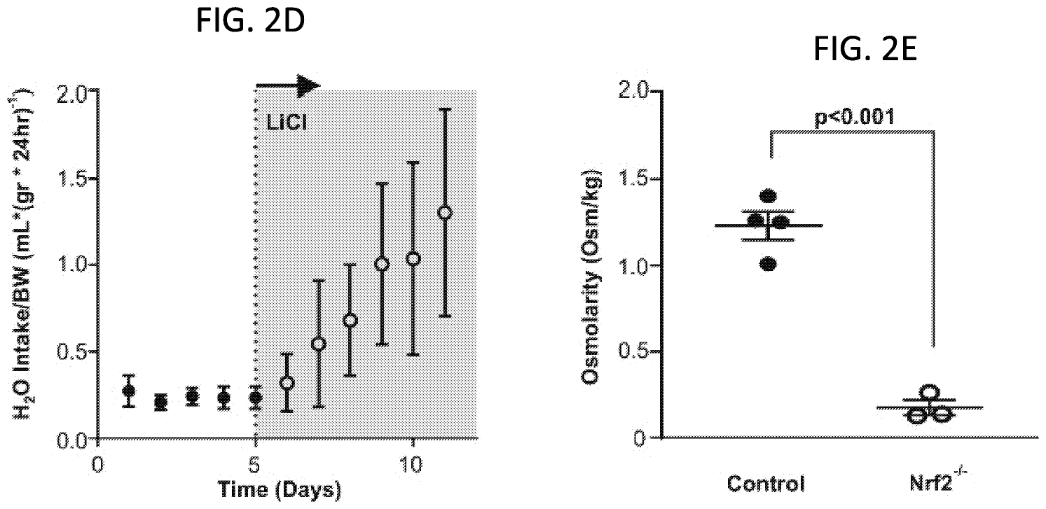

To additionally test if Nrf2 activation is required for Li-NDI development, Li-administration was repeated in mice with global knock-out of Nrf2. Nrf2^{-/-} mice were maintained on control diet for 5 days followed by Li diet for 6 days (FIG. 2A). As in WT, Li slightly reduced body weight (FIG. 2B). Food intake was modestly suppressed in the first 3 days of LiCl diet administration but rebounded to baseline by 4 days (FIG. 2C). Despite Nrf2 ablation, dietary Li induced NDI with temporally similar onset of polydipsia (FIG. 2D) as in wild-type mice (FIG. 1B). Urine osmolality was significantly reduced compared to WT control (176+/−43 mOsm/kg vs. 1230 +/−163 mOsm/kg , p<0.0001) (FIG. 2E), indicating that Nrf2 signaling is not required for development of NDI.

### Example 4

### Graded Activation of Nrf2 Rescues Li-NDI in Mice

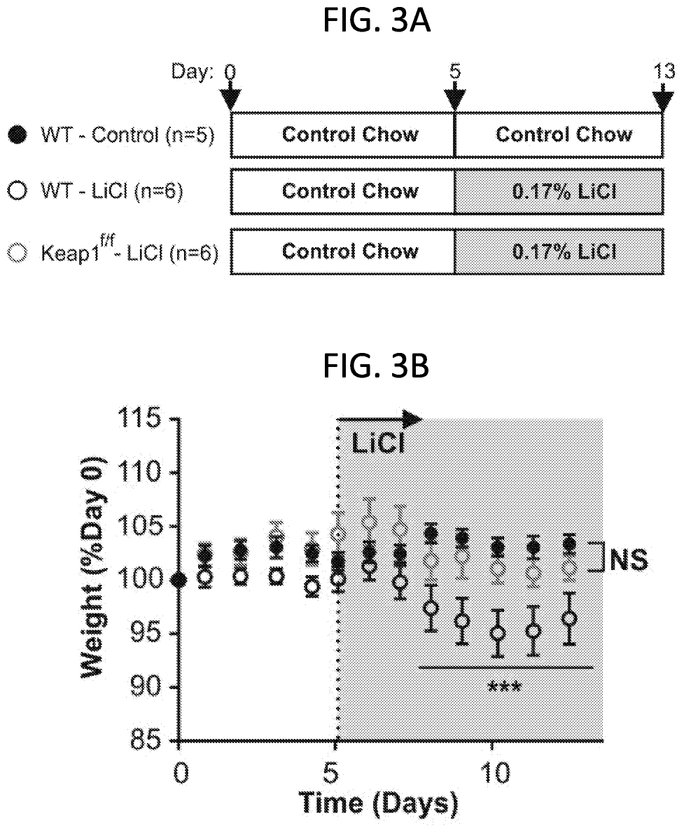

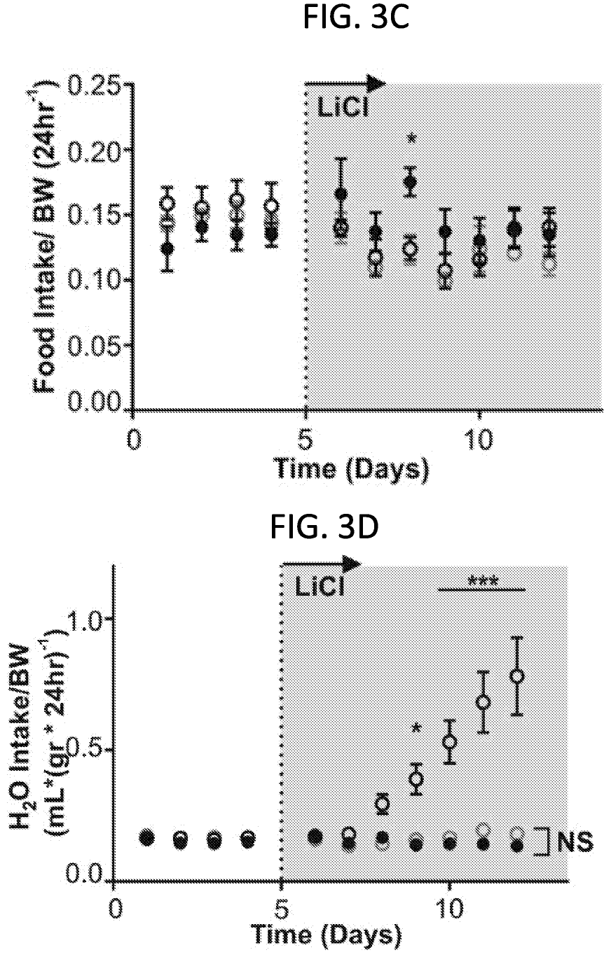

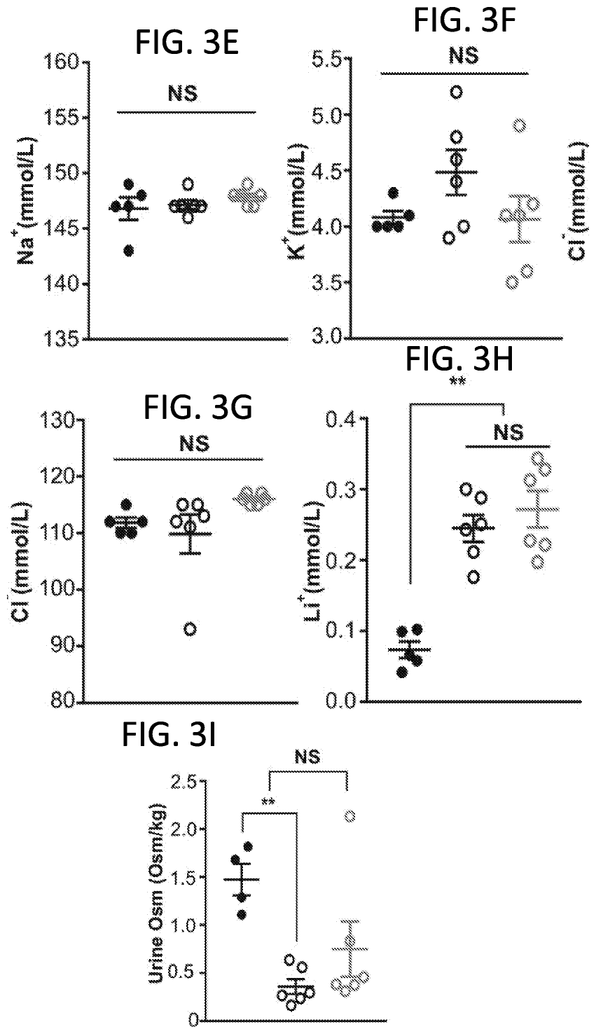

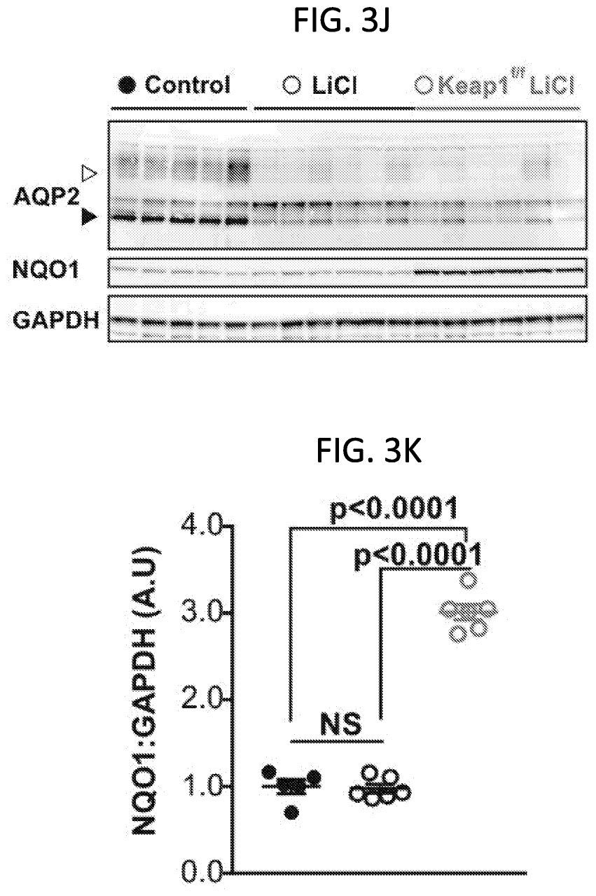

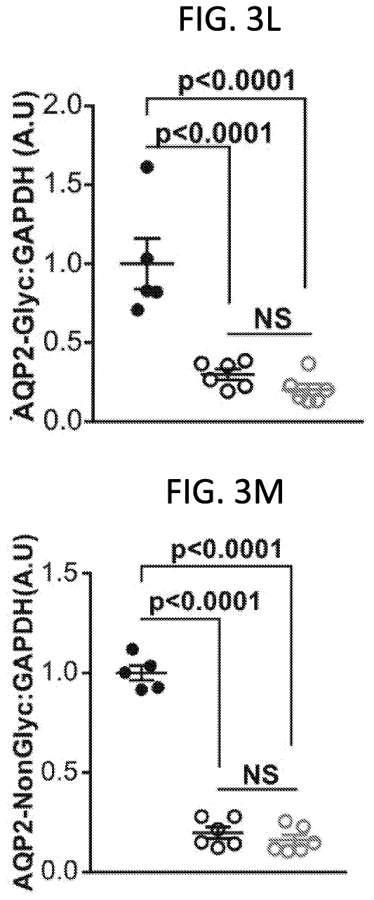

Hyperactivation of Nrf2 through genetic ablation of the E3 ubiquitin ligase complex proteins Keap1 or Cul3 has been shown to induce NDI in mice (Noel et al., BMC Nephrol. 17, (2016); Suzuki et al., Nat. Commun. 8, 14577 (2017); McCormick et al., J. Clin. Invest. 124, 4723-4736 (2014)). Consequently, Li was administered to mice with constitutive pharmacomimetic Nrf2 activation (Keap1^{f/f}) to test whether Nrf2 and Li induced NDI via synergistic or additive mechanisms (FIG. 3A). To our surprise, instead of exacerbating the renal toxicity of Li, activation of Nrf2 signaling conferred significant protective effects. After 3 days, all groups displayed identical food intake, WT-Li mice exhibited modest (˜5%) reduction in body mass animals while Keap1^{f/f} mice receiving LiCl were protected and demonstrated no change when compared to control diet, despite (FIG. 3B-C).

As in our validation studies, Li intake in the WT cohort recapitulated the polydipsia (FIG. 1B, 3D) and correlated with polyuria. Strikingly, Keap1^{f/f} mice receiving Li were normodipsic showing complete protection from development of NDI. Blood chemistry analysis revealed no differences in plasma Na⁺, K⁺, or Cl⁻ between groups suggesting normovolemia (FIG. 3E-G), while plasma Li⁺ was equivalently elevated in both WT and Keap1 ^{f/f} groups (FIG. 3H) suggesting identical absorption, exposure, and clearance of Li.

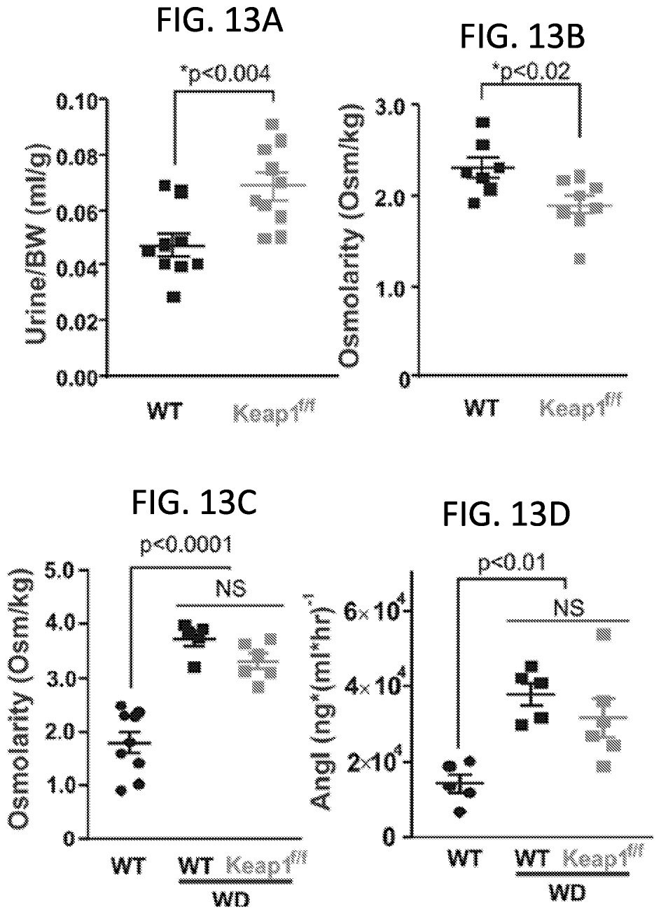

The WT-Li cohort had significantly lower spot urine osmolality than the control diet cohort (359+/−192 mOsm/kg vs 1473+/−332 mOsm/kg , p<0.0001, FIG. 31) indicating that polyuria was accompanied by hyposthenuria consistent with NDI. Despite complete normodipsia, urine osmolality was likewise reduced in the Keap1^{f/f}-Li cohort (758+/−703 mOsm/kg, p=0.08 compared to control and not significantly different from WT-Li) (FIG. 31). Plasma renin concentrations, as a readout for physiological response to plasma osmolality, were identical across experimental groups suggesting that all mice were normovolemic and drinking to satiety (FIG. 13A-B).

Expression of NQO1 was not affected by Li treatment, but was significantly increased in Keap1^{f/f} animals confirming constitutive activation of Nrf2 (FIG. 3J-M). Glycosylated ˜45 kDa (open arrow, FIG. 3J,L) and non-glycosylated 29 kDa (closed arrow, FIG. 3J,M) isoforms of AQP2 were significantly reduced by Li exposure in both groups, consistent with NDI. Surprisingly, AQP2 expression did not correlate with volume intake in Keap1^{f/f} mice.

### Example 5

### Nrf2 Modulates Renal Ion Channel Expression

Clinically, established Li-NDI is treated with inhibitors of the sodium-chloride cotransporter

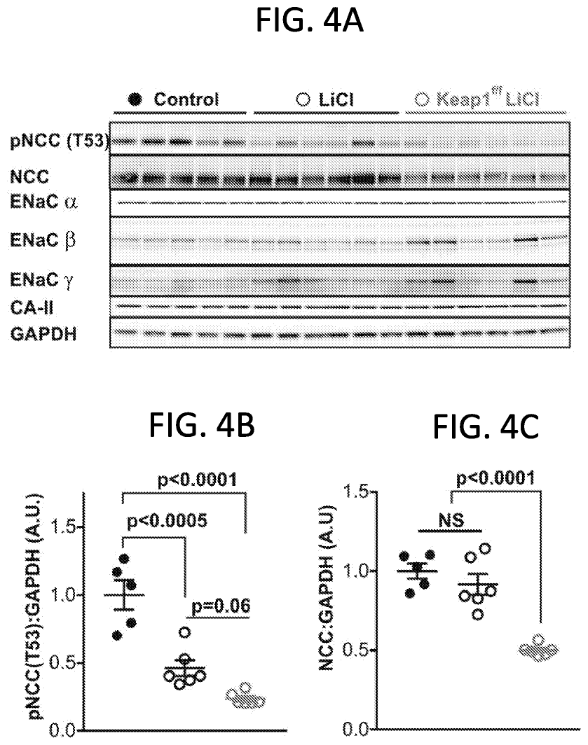

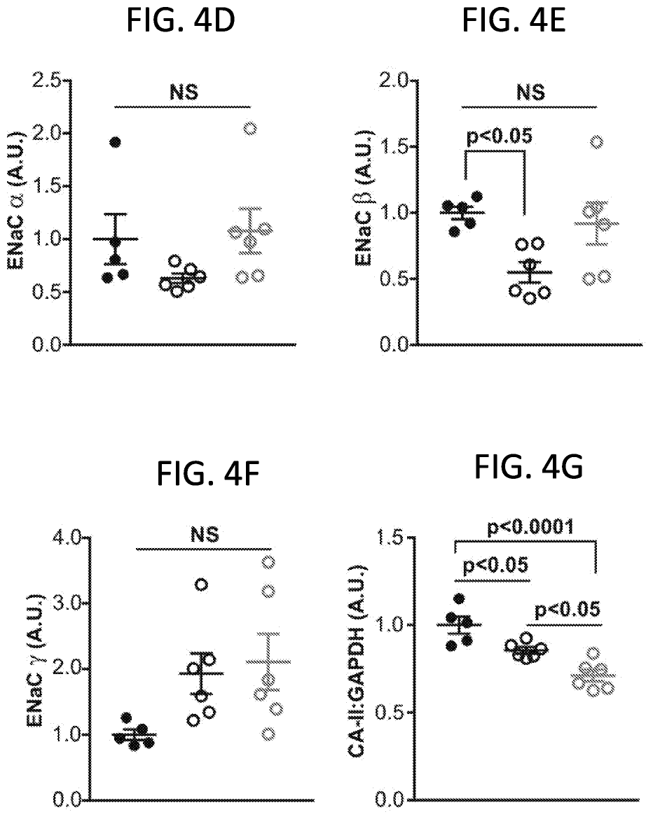

(NCC), epithelial sodium channel (ENaC), carbonic anhydrase (CA), or with NSAIDs which inhibit prostaglandin biosynthesis. Thiazide diuretics exhibit a paradoxical anti-diuretic effect when administered to animals or patients with NDI (Shirley et al., Renal mechanisms. Clin. Sci. 63, 533-538 (1982); Walter et al., Clin. Sci. 63, 525-532 (1982); Konoshita et al., Horm. Res. 61, 63-7 (2004); Kim et al., J. Am. Soc. Nephrol. 15, 2836-2843 (2004); Sinke et al., Am. J. Physiol. Physiol. 306, F525-F533 (2014)). Thus, it was hypothesized that hyperactivation of Nrf2 might modulate abundance or activity of NCC. Activating phosphorylation of NCC at threonine 53 (pNCC, T53) was reduced by Li treatment in both genotypes, while total expression of NCC (tNCC) was unchanged in WT-Li cohort but significantly reduced in Keap1^{f/f} -Li cohort leading to a significant reduction on the pNCC:tNCC ratio in both Li-treated groups (FIG. 4A-C) Amiloride reduces polyuria in murine models of Li-NDI as well as in human patients, through reduction of ENaC mediated uptake of Li (Kosten & Forrest, Am. J. Psychiatry 143, 1563-1568 (1986); Kortenoeven et al. Kidney Int. 76, 44-53 (2009); Christensen et al. J. Am. Soc. Nephrol. 22, 253-261 (2011); Finch et al., Pharmacotherapy 23, 546-550 (2003); Bedford et al., Am. J. Physiol. Physiol. 294, F812-F820 (2008); Bedford et al., Clin. J. Am. Soc. Nephrol. 3, 1324-31 (2008); Kalita-De Croft et al., Nephrology 23, 20-30 (2018)). No differences were observed between expression of ENaC subunits α, β, or γ suggesting that regulation of this transporter was not involved in Nrf2-mediated resistance to NDI (FIG. 4D-F). Likewise, Li reduced expression of another therapeutic target, CA-II (de Groot et al., Am. J. Physiol. Physiol. 313, F669-F676 (2017); de Groot et al., J. Am. Soc. Nephrol. 27, 2082-2091 (2016); Gordon et al., N. Engl. J. Med. 375, 2008-2009 (2016)), in WT animals, with an additional reduction in Keap1^{f/f} -Li mice (FIG. 4G). Collectively, these data suggest that protection from Li-NDI in Keap1^{f/f} mice is at least partially due to reduced NCC and CA-II activity.

### Example 6

### Keap1 Hypomorphism Induces Phenotype Distinct From Complete Global Or Kidney-Specific Knockout

The resistance of Keap1^{f/f} mice to Li-NDI was an unexpected finding, as recent studies have shown that genetic hyperactivation of Nrf2 through total ablation of renal Keap1 causes NDI (Noel et al., BMC Nephrol. 17, (2016); Suzuki et al., Nat. Commun. 8, 14577 (2017)). Down-regulation of solute transporters such as NCC in Keap1^{f/f} mice would additionally suggest impaired ion reabsorption and increased diuresis. Indeed, under basal conditions, Keap1^{f/f} mice were found to be mildly polyuric and hyposthenuric (Suppl. FIG. 4A-B). However, upregulation of plasma renin was normal and urine concentration in response to 12-hour water deprivation were no different from WT (FIG. 13C-D). This indicates that while kidney function is markedly impaired by complete ablation of Nrf2 repressors (Noel et al., BMC Nephrol. 17, (2016); Suzuki et al., Nat. Commun. 8, 14577 (2017); McCormick et al., J. Clin. Invest. 124, 4723-4736 (2014)), Nrf2 graded activation in Keap1^{f/f} does not impair concentrating ability in our murine model.

### Example 7

### Renal Effects of Graded Genetic and Pharmacologic Activation of Nrf2 are Localized to the Distal Tubule

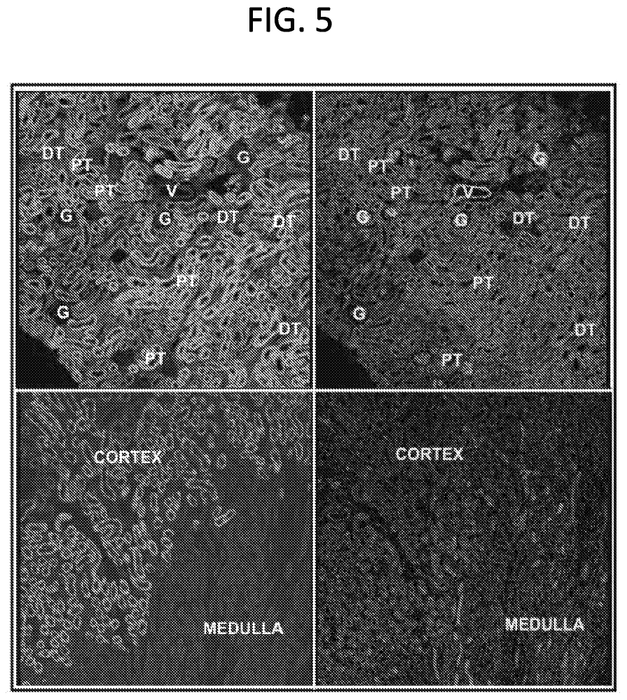

Surprisingly, despite extensive research evaluating Nrf2 as a pharmacologic target for renal diseases, the distribution and sensitivity of Nrf2 signaling activity in the kidney has not been characterized Immunofluorescence microscopy was performed on kidney sections from WT mice probing for the Nrf2 target NQO1 and the marker Mucin 1 (MUC1) which is expressed in the distal convoluted tubule, connecting tubule, and collecting system (Aubert et al. Cancer Res. 69, 5707-15 (2009); Braga et al., Development 115, 427-37 (1992); Pastor-Soler et al., Am. J. Physiol. Physiol. 308, F1452—F1462 (2015)). NQO1 was found to be highly expressed in the renal cortex with localization to the proximal tubules (PT), which stained negative for MUC1. By contrast, glomeruli (G), vessels (V), MUC1-positive distal/connecting tubules (DT/CT), and the renal medulla displayed low expression of NQO1 (FIG. 5A).

NQO1 staining of kidneys from Keap1^{f/f} mice showed upregulation of NQO1 in tubule segments with low Nrf2 activity in WT counterparts, but not glomeruli or vessels. Significantly, co-expression of MUC1 and NQO1 was observed in cortex suggesting increase of Nrf2 activity in DT/CT. NQO1 abundance was found to be increased in both the outer and inner renal medulla of Keap1^{f/f} animals (FIG. 5B). To further test the distribution of Nrf2 and sensitivity to activation in a translational model, NQO1 expression was determined in a cell culture model systems of primary human renal cortical cells representing proximal tubule (PT) and distal tubule (DT) cell populations (Emlet et al., Am. J. Physiol. - Ren. Physiol. 312, (2017)) in the presence or absence of the Nrf2 activator CDDO-Imidazolide (CDDO-Im) (FIG. 5C-F). Supporting the observations made in mouse, NQO1 expression was significantly higher in proximal tubule than in distal tubule. Recapitulating the effects of genetic activation of Nrf2 in Keap1^{f/f} mice, NQO1 expression did not further increase upon exposure to CDDO-Im in CD-10/CD-13 positive HAK cells, whereas the MUC1 positive cells responded robustly with 3-fold elevation in NQO1. Taken together, these observations support a model of high basal Nrf2 activity in the proximal tubule and sensitivity to genetic or pharmacologic induction in the distal convoluted tubule and collecting system (FIG. 5G).

### Example 8

### Nrf2 Modulates Vascular Reactivity Through Decreased Prostaglandin Biosynthesis

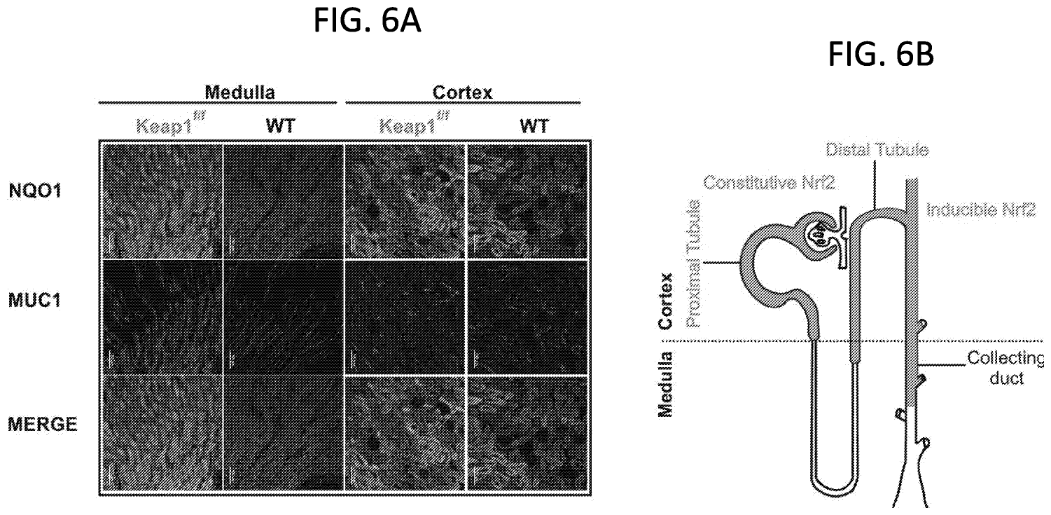

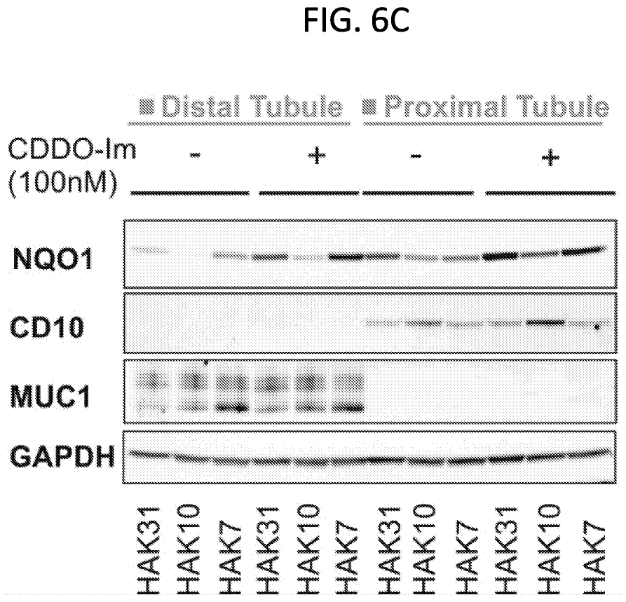

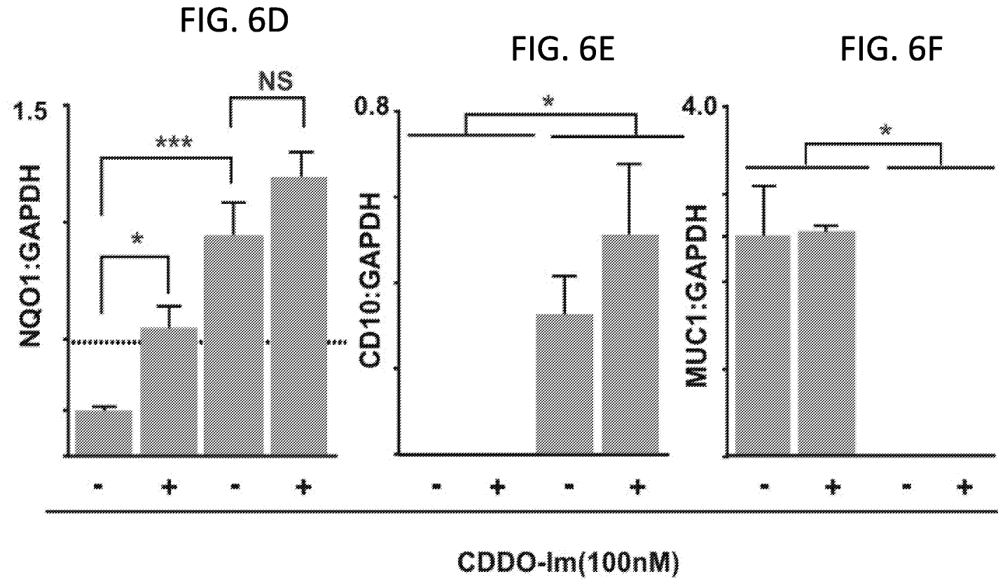

While the exact mechanism remains unclear, paradoxical protection afforded by diuretics in Li-NDI is thought to involve tubuloglomerular feedback (TGF) leading to a reduction of glomerular filtration rate (GFR). Thiazides and acetazolamide dramatically diminish polyuria induced by Li but have only marginal effects on AQP2 abundance and urine osmolality (Kim et al., J. Am. Soc. Nephrol. 15, 2836-2843 (2004); Sinke et al., Am. J. Physiol. Physiol. 306, F525-F533 (2014); de Groot et al., Am. J. Physiol. Physiol. 313, F669-F676 (2017); de Groot et al., J. Am. Soc. Nephrol. 27, 2082-2091 (2016)). To test the effect of Nrf2 activity on vascular function, wire myography studies were performed on isolated mesenteric and thoracodorsal arteries from WT and Keap1^{f/f} mice. Vessels from Keap1 ^{f/f} mice showed decreased sensitivity and blunted responses to the vasodilator acetylcholine, indicating endothelial dysfunction (FIG. 6A-B). The EC50 of ACh was significantly reduced in both vessel types in Keap1 ^{f/f} animals, but phenylephrine-induced vasoconstriction was unaffected (FIG. 6C).

Dilation of resistance vessels is modulated by effects of paracrine relaxing factors produced by the vascular endothelium on vascular smooth muscle cells. To determine the mechanism underlying blunted vasodilation and increased vascular resistance, the nitric oxide (NO) signaling pathway was evaluated. Surprisingly, plasma and urine NO₂⁻ (surrogate for NO formation) were significantly elevated in Keap1^{f/f} animals (FIG. 6D-E). Plasma arginine, the precursor from which NO is produced, was equally abundant in Keap1^{f/f} mice as in WT counterparts (FIG. 6F). Plasma asymmetric dimethylarginine (ADMA), an inhibitor of eNOS (Vallance et al., Lancet 339, 572-5 (1992)), was likewise unchanged. Expression and activating phosphorylation of endothelial nitric oxide synthase (eNOS) in kidney homogenates was normal in Keap1^{f/f} mice, and nNOS was detected in brain tissue (positive control) but not in kidney lysates (FIG. 6G-H). Renal expression of the NO signal transducer soluble guanylate cyclase β1 (sGC-β1) was marginally lower in Keap1^{f/f} than in WT mice, however this was not physiologically significant as the sGC-β1 product cGMP was found to be slightly higher but not statistically different in plasma from Keap1^{f/f} mice (FIG. 6I). Finally, vasodilation in response to the chemical NO-donor sodium nitroprusside (SNP) was identical between WT and Keap1^{f/f} mice, indicating that impaired vasodilation was not caused by reduced sensitivity to NO (FIG. 6J). Together, these results indicate that Nrf2 hyperactivation inhibits ACh-induced vasodilation independently of NO production or downstream signaling, and elevated plasma and urine nitrite together with plasma cGMP suggest that this pathway may be hyperactivated to compensate for a defect in an alternate pathway.

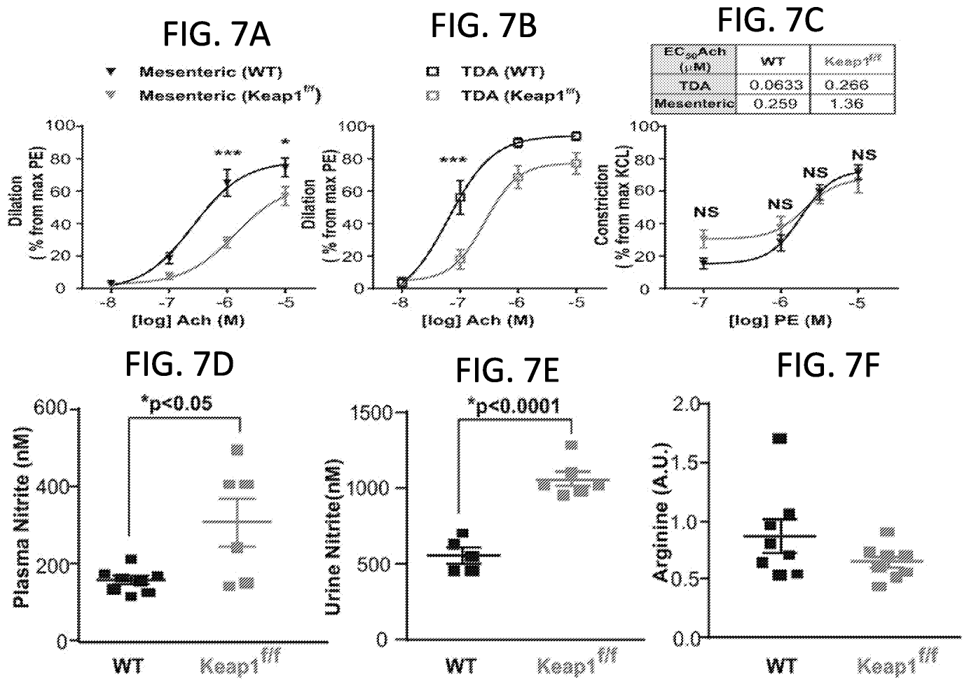

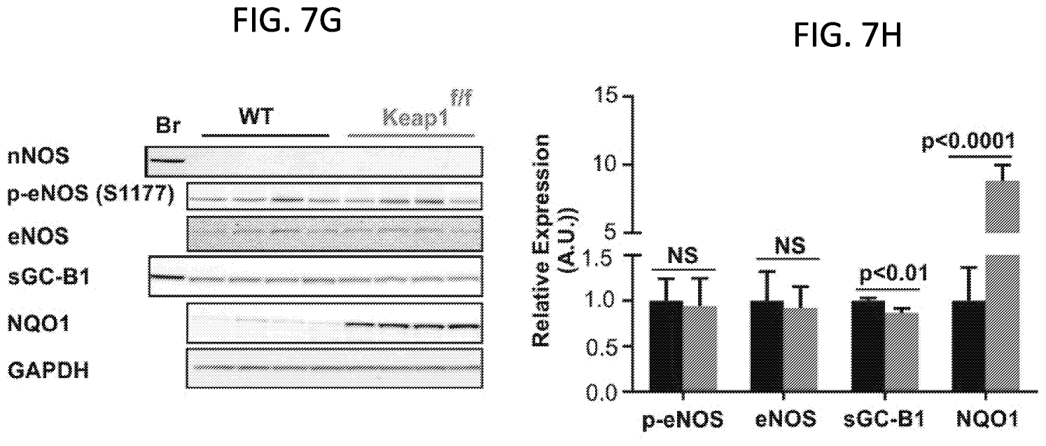

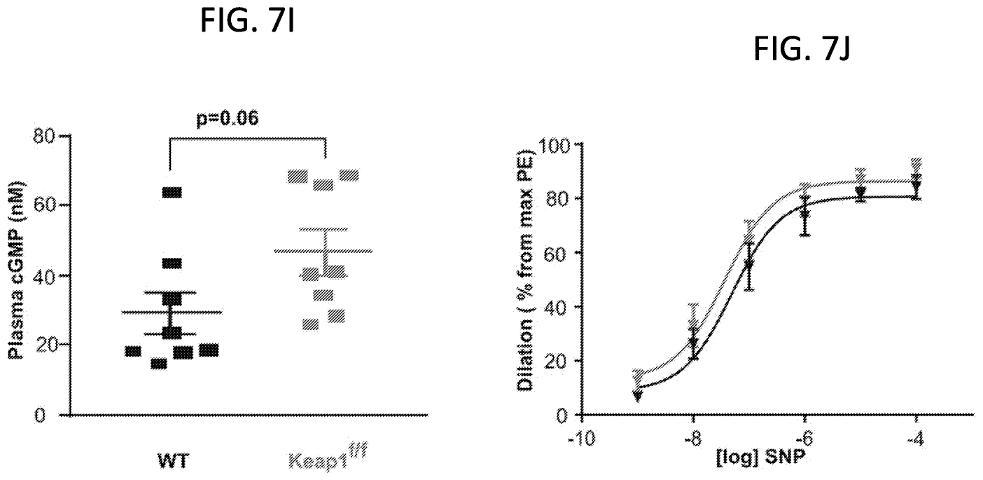

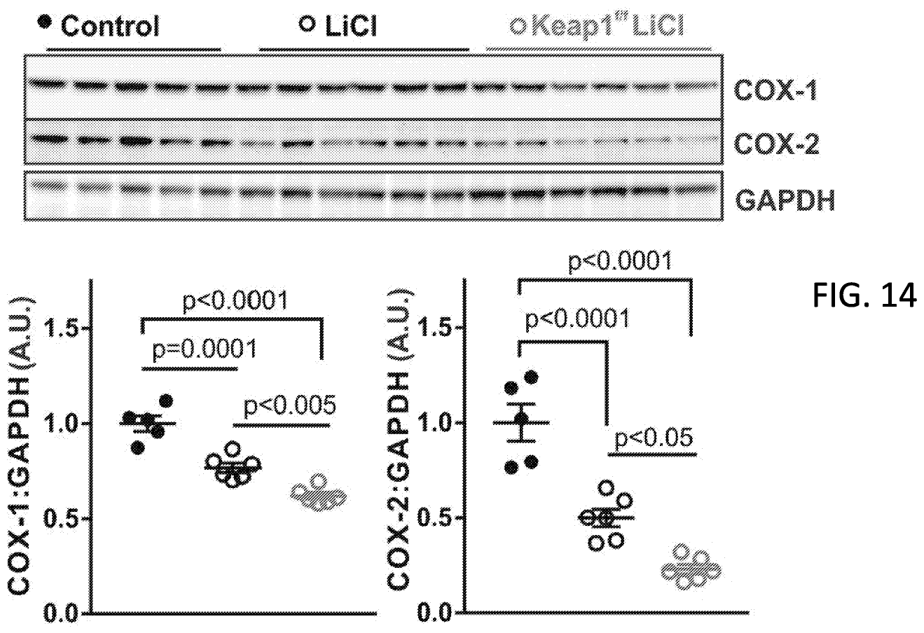

Tubuloglomerular filtration (TGF) is a direct result of paracrine and endocrine signaling from the macula densa at the apex of the ascending limb of the loop of Henle to induce vasoconstriction of afferent arterioles. Both the renin-angiotensin-aldosterone signaling (RAAS) and prostaglandin biosynthesis have been implicated in this process. To test the role of the renin-angiotensin-aldosterone signaling (RAAS) in Nrf2 protection against Li-NDI, plasma renin and prostaglandin and renal COX expression were evaluated. Plasma renin activity was unchanged between WT and Keap1^{f/f} mice after water deprivation (FIG. 13D) or after receiving Li (FIG. 14A), indicating that the effects of Nrf2 could be downstream of Angiotensin formation and involve prostaglandin synthesis. COX-1 and COX-2 expression were found to be reduced in Keap1^{f/f}-Li mice (FIG. 14B) suggesting that Nrf2 activation has direct effects on renal expression of these proteins. Because Li was additionally found to down-regulate COX-1 and COX-2 in WT mice, the experiment was repeated comparing tissues from WT and Keap1^{f/f} mice on control diet, which confirmed significant reduction in renal COX-1 and COX-2 by Nrf2 (FIG. 7A-B). Down-regulation of COX-2 in Keap1^{f/f} mice was further confirmed by immunohistochemical staining of kidney sections (FIG. 7C). Additionally, kidneys from Nrf2^{-/-} animals showed increased COX-1 and COX-2 expression compared to control (FIG. 7D-E), confirming regulation of COX-1 and COX-2 by Nrf2 signaling. The downregulation of COX expression in Keap1^{f/f} mice correlated with a reduction in the renal levels of a stable prostacyclin (PGI₂) metabolite, 6-keto-PGF_{1α}, (FIG. 7F). In addition, kynurenine, a recently discovered endothelium-derived relaxing factor derived from inflammation-related pathways that acts through activation of adenylate and soluble guanylate cyclase pathways (Pawlak et al., Atherosclerosis 204, 309-314 (2009); Wang et al. Nat. Med. 16, 279-85 (2010)) was significantly reduced compared to WT, paralleling the reduction in 6-keto-PGF_{1α}, (FIG. 7G). Together these results suggest that effects of constitutive Nrf2 activation on inflammation-related autocoid production impact vascular compliance and cardiovascular homeostasis, offering a putative mechanism for resistance to Li-NDI.

### Example 9

### Pharmacologic Activation of Nrf2 with CDDO-Me Protects Against Li-NDI

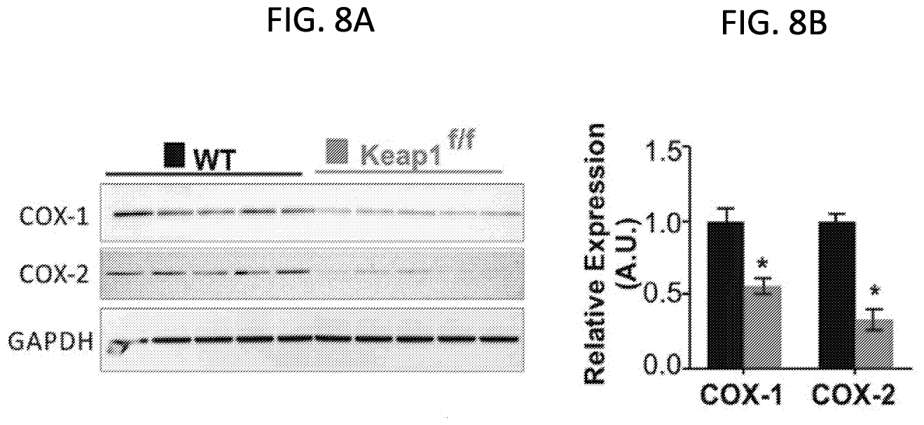

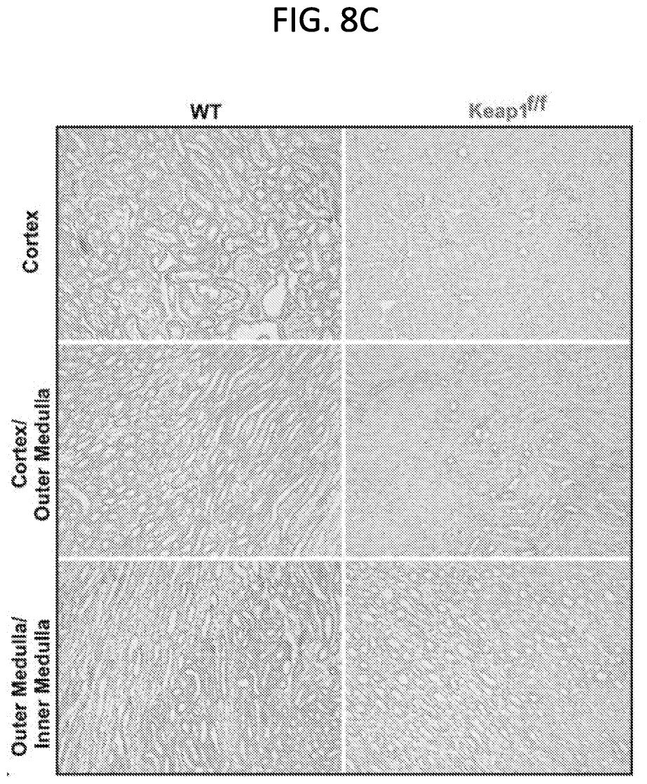

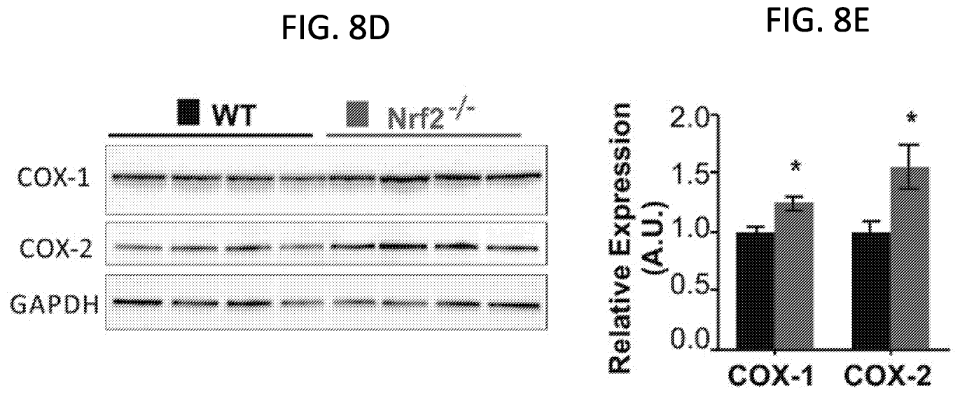

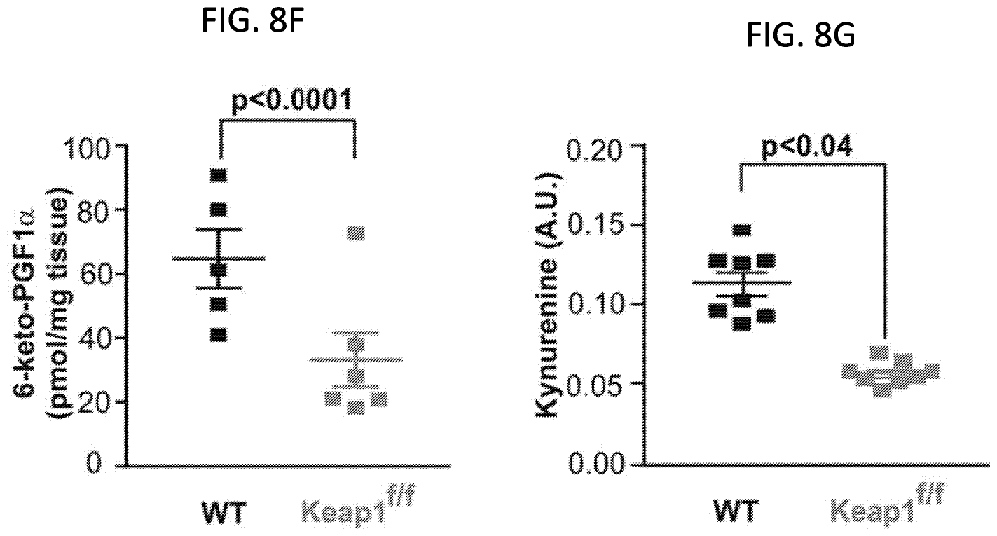

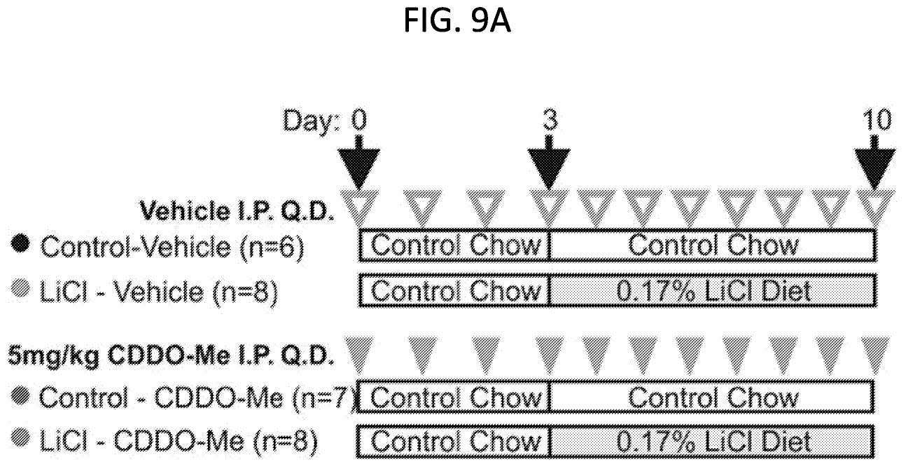

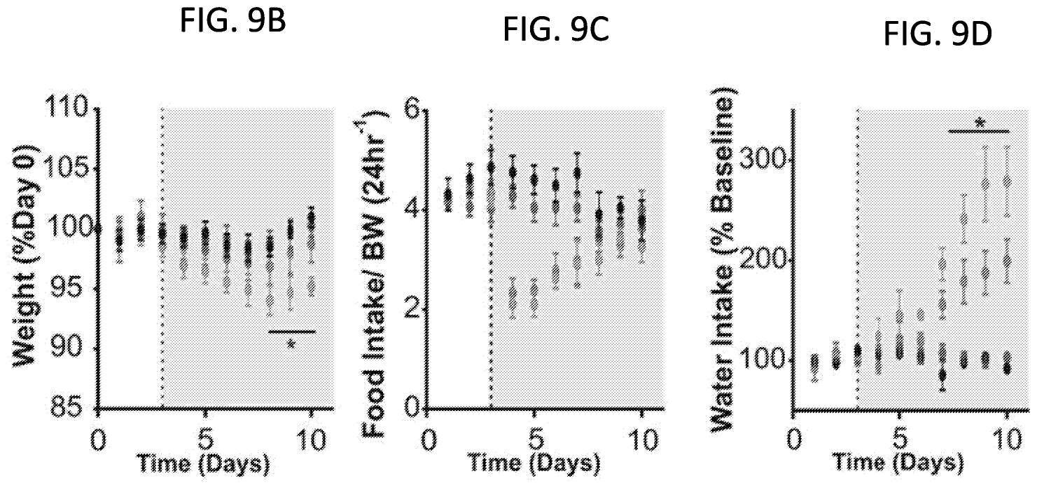

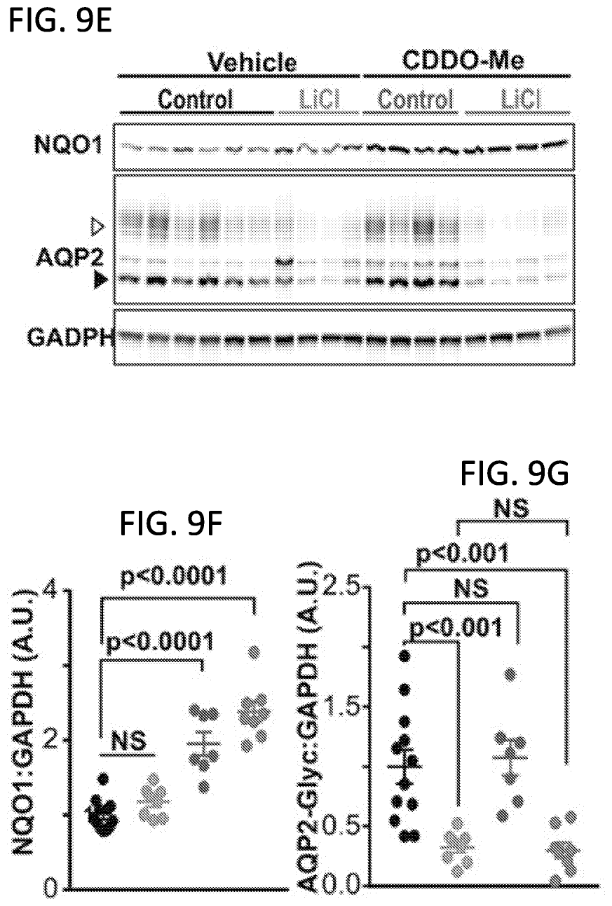

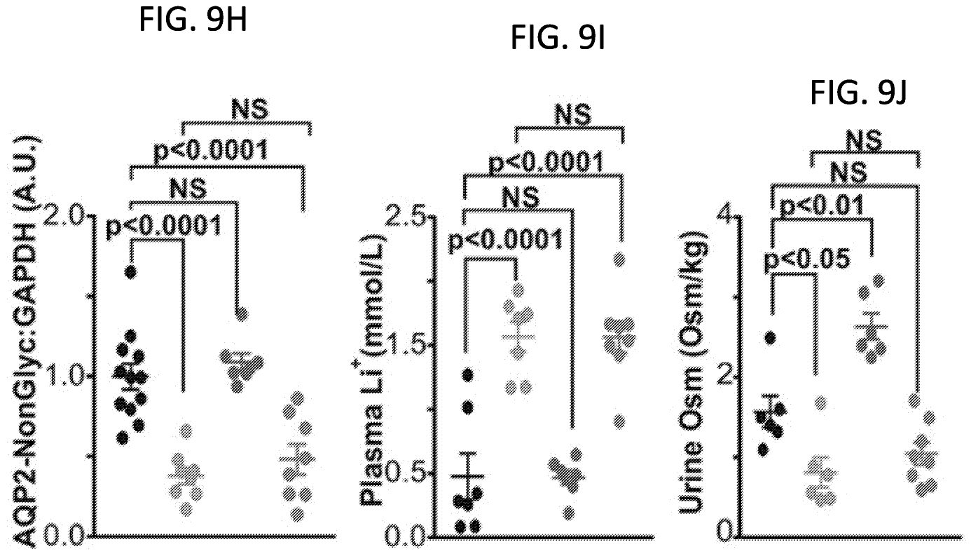

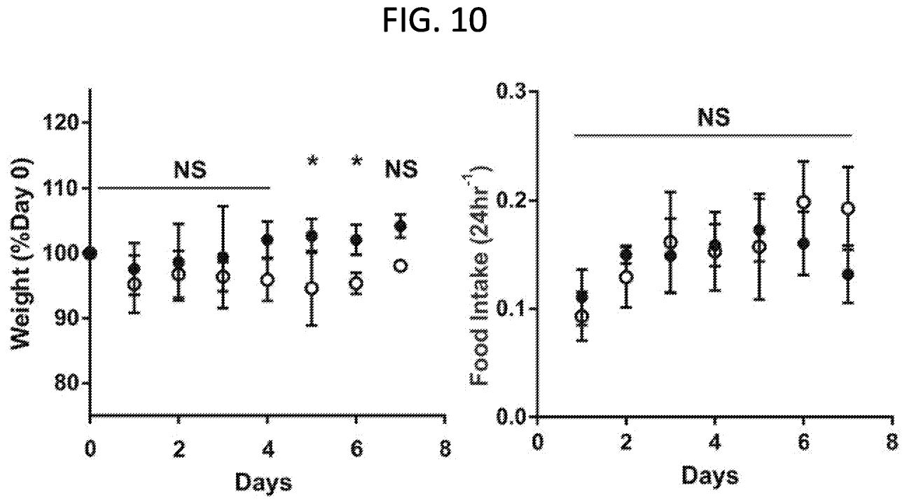

The strong protective effect of genetic Nrf2 activation in Li-NDI motivated evaluation of the viability of pharmacologic Nrf2 activation as a therapeutic intervention. Bardoxolone methyl (CDDO-Me) is a synthetic triterpenoid electrophile which potently activates Nrf2 and is in Phase II/III clinical trials for treatment of Alport Syndrome, a genetic disease characterized by progressive loss of kidney function (Reata Pharmaceuticals. A Phase 2/3 Trial of the Efficacy and Safety of Bardoxolone Methyl in Patients With Alport Syndrome-CARDINAL (CARDINAL). National Library of Medicine (US) Availa), as well as other rare chronic kidney diseases (Reata Pharmaceuticals. A Phase 2 Trial of the Safety and Efficacy of Bardoxolone Methyl in Patients With Rare Chronic Kidney Diseases-PHOENIX (PHOENIX). National Library of Medicine (US) Available at: https://clinicaltrials.gov/ct2/show/NCT03366337 (Accessed: 30th Oct. 2018)). Administration of CDDO-Me beginning 3 days prior to Li and continuing throughout Li exposure (FIG. 8A) significantly reduced water intake and protected against weight loss without impacting food consumption (FIG. 8B-D) or reducing plasma Li⁺ (FIG. 81). Similar to genetic Nrf2 hyperactivation via Keap1 hypomorphism, CDDO-Me increased renal NQO1 expression 2- to 3-fold (FIG. 8E-F). AQP2 abundance and glycosylation were not improved by CDDO-Me (FIG. 8G-H), and urine osmolality showed only marginal improvement (FIG. 8J). This demonstrates that pharmacologic Nrf2 activation, similar to genetic activation by Keap1 hypomorphism, protects against Li-NDI via mechanisms unrelated to AQP2 expression.

### Example 10

### Activation Of Nrf2 With 10-Nitro-9(E)-Octadec-9-Enoic Acid Reduces Symptoms of NDI

The pharmacologic activation of Nrf2 with CDDO-Me provided motivation to evaluate the viability of 10-nitro-9(E)-octadec-9-enoic acid as an activator and potential API for treating or preventing NDI. The compound of 10-nitro-9(E)-octadec-9-enoic acid reduced symptoms of nephrogenic diabetes insipidus.

### Example 11

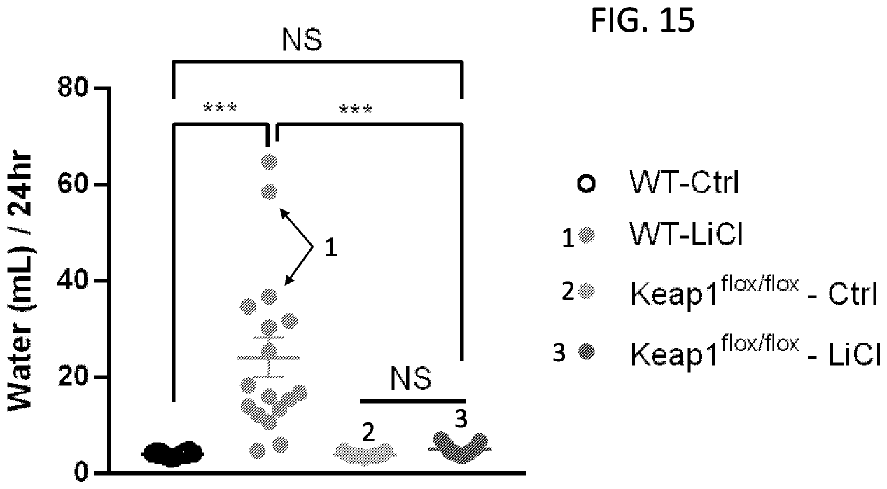

The duration of Li therapy in humans is on the order of months to decades. An experiment was done to evaluate if Nrf2 hyperactivation conferred long-term protection from the development of Li-NDI. 6-8 week old WT and Keap1^{flox/flox} mice were randomized to control or 0.17% LiCl diet for 8 months. Nrf2 mediated protection remained complete after 8 months of chronic lithium exposure, with no significant increase in average daily water intake in Keap1^{flox/flox} -Li mice compared to WT-Ctrl (FIG. 15).

Lithium has remained a mainstay drug for mood stabilization in bipolar disorder for over a half-century and is increasingly re-purposed for treatment of other CNS diseases as new therapeutic effects are described and mechanisms documented. Disparagingly, the beneficial effects of Li are offset by adverse functional and structural renal sequelae. In addition to causing polyuria (>3,000 mL urine/day), chronic Li therapy may promote or cause development of CKD (Aiff et al., 24, 540-544 (2014); Rabin et al., Can. Med. Assoc. J. (1979); Cairns et al., Br. Med. J. (Clin. Res. Ed). (1985). doi:10.1136/bmj.290.6467.516; Garofeanu et al., American Journal of Kidney Diseases (2005). doi:10.1053/j.ajkd.2005.01.008; Aiff et al.,J. Psychopharmacol. 29, 608-614 (2015)). Furthermore, Li has a narrow therapeutic index and is renally excreted; CKD or other kidney injury complicates maintenance of plasma levels within a therapeutic range. As no alternatives match Li in efficacy of acute and chronic management and reduction of suicide risk in bipolar disorder (Song et al. S Am. J. Psychiatry 174, 795-802 (2017)), discontinuation poses a significant clinical challenge for nephrologists and psychiatrists caring for patients with this disease (Goodwin, JAMA Psychiatry 72, 1167 (2015)). 75% of patients who are stable on Li have recurrent mood episodes within 5 years after discontinuation (Faedda et al., Arch. Gen. Psychiatry (1993). doi:10.1001/archpsyc.1993.01820180046005), and frequently require psychiatric hospitalization.

Recent evidence implicates the Keap1/Nrf2 signaling pathway as playing a role in regulating AQP2 via as-of-yet unknown mechanisms. Specifically, hyperactivation of Nrf2 signaling by ablation of its repressors Cul3, GSK3β, and Keap1 have independently been found to cause NDI in mice; (Noel et al., BMC Nephrol. 17, (2016); Suzuki et al., Nat. Commun. 8, 14577 (2017); McCormick et al., J. Clin. Invest. 124, 4723-4736 (2014); Rao et al., J. Am. Soc. Nephrol. 21, 428-37 (2010)). As Li is an inhibitor of GSK3β, it was hypothesized that Li induces NDI via hyperactivation of Nrf2. The results present herein demonstrate that NDI develops rapidly during dietary Li administration, with significant increase in water intake and polyuria/hyposthenuria secondary to reduction in AQP2 expression. Despite developing a robust NDI phenotype, Li-treated mice did not display engagement of Nrf2 signaling in the kidney and Nrf2^{-/-} mice developed NDI similarly to WT mice, suggesting that hyperactivation of this pathway was not involved.

Transgenic mice with total ablation of Keap1 in the kidney epithelium (Noel et al., BMC Nephrol. 17, (2016)) or whole animal (NEKO) (Suzuki et al., Nat. Commun. 8, 14577 (2017) (Suzuki et al., Nat. Commun. 8, 14577 (2017)) develop NDI. Surprisingly, it was found that mice with Keap1 hypomorphism are protected against development of Li-NDI, with complete normalization of water intake compared to WT mice receiving Li. In comparison to Keap1 total-knock-out animals, which exhibit a range of developmental abnormalities (Noel et al., BMC Nephrol. 17, (2016); Suzuki et al., Nat. Commun. 8, 14577 (2017); Wakabayashi et al., Nat. Genet. 35, 238-245 (2003); Yoshida, E. et al., Genes to Cells (2018). doi:10.1111/gtc.12579), hypomorphism via floxing of exons 4-6 with loxP sites leads to pharmaco-mimetic activation and tissue protection (Taguchi et al. Mol. Cell. Biol. 30, 3016-3026 (2010)). In striking contrast to Keap1^{-/-} animals, Keap1^{f/f} mice exhibited only mild hyposthenuria and polyuria at baseline, and had normal urine concentrating ability and upregulation of plasma renin activity in response to 12 hr water deprivation. It is possible that Nrf2 activation displays hormesis, with activity above a certain threshold causing adverse outcomes.

Kidneys maintain solute and fluid homeostasis through a complex sequence of passive and active transport mechanisms localized to specific nephron segments. The kidney has high energy requirements due to significant active transport mechanisms required for movement of solutes against their concentration gradients as well as the processes of detoxification of reactive compounds through conjugation and excretion. Indeed, despite their small size (˜0.5% body weight) the kidneys consume about ˜10% of total oxygen used in cellular respiration. A gradient of glucose availability establishes from the renal cortex inwards resulting in low O₂ tensions in the inner medulla and a predominantly anaerobic metabolism (Chen et al., Bull. Math. Biol. 78, 1318-1336 (2016)). In the context of the kidneys' high energetic demands and its xenobiotic exposure, the protective role of Nrf2 gains significance. The spatial distribution NQO1 expression in murine kidney appears to parallel the postulated O₂ and glucose gradients. Moreover, it appears that in both mouse and man, Nrf2 activity is significantly higher in proximal than in distal tubule epithelium. The proximal tubules are responsible for the bulk of solute, water, and small-molecule reabsorption as well as for conjugation and excretion of toxins and wastes (Curthoys & Moe, Clin. J. Am. Soc. Nephrol. 9, 1627-38 (2014)). Previously, human primary proximal tubule cells in culture have been found to express membrane transporters and conjugation enzymes required for these processes (Lash et al., Toxicology 228, 200-218 (2006); Lash et al.Toxicology 244, 56-65 (2008)), many of which are under Nrf2 transcriptional control (Hayes, & Dinkova-Kostova, Trends in Biochemical Sciences 39, 199-218 (2014)). While at baseline NQO1 expression was significantly lower in distal tubule epithelial cells than proximal tubule epithelial cells, distal tubule cells were more sensitive to both genetic activation and the electrophilic Nrf2 inducer CDDO-Im. Consistent with these observations, protein abundance of the DT marker NCC was reduced in kidneys from Keap1^{f/f} mice suggesting, without being bound by theory, that upregulation of Nrf2 might impact cell differentiation, given the plasticity shown by renal epithelial cells (Park, J. et al., bioRxiv 203125 (2017). doi:10.1101/203125).

The renal damage caused by prolonged Li therapy is reversible in its early stages but may progress to irreversible deleterious remodeling and loss-of-function (Rabin et al., Can. Med. Assoc. J. (1979); Cairns et al., Br. Med. J. (Clin. Res. Ed). (1985). doi:10.1136/bmj.290.6467.516; Garofeanu et al., American Journal of Kidney Diseases (2005). doi:10.1053/j.ajkd.2005.01.008). Risk of progression to end-stage renal disease is significantly greater in patients on Li than in the general population, indicating a substantial unmet clinical need (Close, H. et al. PLoS One 9, e90169 (2014); Aiff et al., 24, 540-544 (2014)). Existing therapeutic approaches for treating Li-NDI fall under two main categories: (1) diuretics or (2) inhibition of renal COX-1 and/or COX-2 with NSAIDs. The diuretics acetazolamide (de Groot et al., Am. J. Physiol. Physiol. 313, F669-F676 (2017); de Groot et al., J. Am. Soc. Nephrol. 27, 2082-2091 (2016); Gordon et al., N. Engl. J. Med. 375, 2008-2009 (2016)), amiloride (Kosten & Forrest, Am. J. Psychiatry 143, 1563-1568 (1986); Kortenoeven et al. Kidney Int. 76, 44-53 (2009); Christensen et al. J. Am. Soc. Nephrol. 22, 253-261 (2011); Finch et al., Pharmacotherapy 23, 546-550 (2003); Bedford et al., Am. J. Physiol. Physiol. 294, F812-F820 (2008); Bedford et al., Clin. J. Am. Soc. Nephrol. 3, 1324-31 (2008); Kalita-De Croft et al., Nephrology 23, 20-30 (2018)), furosemide (Michimata, M. et al., Kidney Int. 63, 165-171 (2003)), and hydrochlorothiazide (Shirley et al., Renal mechanisms. Clin. Sci. 63, 533-538 (1982); Walter et al., Clin. Sci. 63, 525-532 (1982); Konoshita et al., Horm. Res. 61, 63-7 (2004); Kim et al., J. Am. Soc. Nephrol. 15, 2836-2843 (2004); Sinke et al., Am. J. Physiol. Physiol. 306, F525-F533 (2014)) have been found to paradoxically reduce urine output in Li-NDI. COX inhibitors have been used as a last-line therapeutic (Allen et al., Arch. Intern. Med. 149, 1123 (1989); Kim et al., Am. J. Physiol. Physiol. 294, F702-F709 (2008)). However use of these compounds is contraindicated in patients with CKD due to potential exacerbation of hypoperfusion injury. While all of these interventions have been documented to reduce polyuria/polydipsia, the underlying mechanisms remain unclear. For instance, while in some studies acetazolamide increased cortical collecting duct abundance of AQP2 (de Groot et al., J. Am. Soc. Nephrol. 27, 2082-2091 (2016)), the reduction of polyuria is independent of AQP2 expression levels (de Groot et al., Am. J. Physiol. Physiol. 313, F669-F676 (2017)). Similarly, thiazides were long thought to paradoxically reduce polyuria through their inhibition of NCC, however, recent evidence shows that this class of drugs also mitigates Li-NDI in NCC knockout animals (Sinke et al., Am. J. Physiol. Physiol. 306, F525-F533 (2014)). With the exception of amiloride, AQP2 protein abundance is not significantly increased by any of these treatments. Furthermore, while these diuretics rescue polyuria/polydipsia they improve maximal urine osmolality only minimally, suggesting that their site of action is not the cortical collecting duct. Based on this evidence, the protection is thought to involve modulation of tubuloglomerular feedback in conjunction with proximal tubule water reabsorption. Reduction of distal sodium reabsorption is believed to deplete extracellular volume and lead to reduction in glomerular filtration rate (GFR) as well as increasing proximal sodium and water reabsorption through effects on medullary osmolality and proximal tubule function (Magaldi, Nephrol. Dial. Transplant. 15, 1903-1905 (2000)).

The phenotype displayed by Keap1^{f/f} mice receiving Li mimicked aspects of the therapies reported in the studies discussed above. While Nrf2 hyperactivation completely prevented polyuria, the urine produced was dilute and expression of both glycosylated and non-glycosylated AQP2 was significantly reduced compared to WT control, and no different from WT-Li. Expression of both NCC and CA-II was modestly down-regulated, suggesting that distal Na⁺ reabsorption may be reduced. Reduction of distal tubular reabsorption with HCTZ [Please provide full name.] has been postulated to promote proximal tubular reabsorption to attenuate polyuria (Konoshita et al., Horm. Res. 61, 63-7 (2004)).

Genetic Nrf2 activation and pharmacologic targeting of Nrf2 have been shown to be protective in murine models of vasculopathy (Li et al., Thromb. Vasc. Biol. 29, 1843-1850 (2009); Howden, Oxidative Medicine and Cellular Longevity (2013). doi:10.1155/2013/104308; Qin, Q. et al., Hypertension 67, 107-117 (2016)). However, the effects of Nrf2 activation on vascular physiology have not been previously studied. In this context, the molecular mechanisms responsible for myogenic control of renal function were evaluated. Resistance arteries isolated from WT and Keap1^{f/f} mice revealed that hyperactivation of Nrf2 caused changes in endothelial function with reduced vasodilation in response to acetylcholine. The vascular effects of Nrf2 activation occurred independently of NO signaling.

The anti-inflammatory effects of Nrf2 activation have been the subject of extensive study and have motivated pharmacologic development of Nrf2 inducers for the treatment of a variety of disorders (Ahmed et al., Biochimica et Biophysica Acta-Molecular Basis of Disease 1863, 585-597 (2017)). The interplay between Nrf2 and inflammatory pathways is complex and involves a direct interaction between Nrf2 accessory proteins such as Keap1 and the nuclear factor kappa B (NF-KB) (Morgan & Liu, Cell Res. 21, 103-15 (2011); Wardyn et al. Biochem. Soc. Trans. 43, 621-6 (2015)) and direct protection by Nrf2 from oxidative stress during inflammation. In addition, Nrf2 reduces pro-inflammatory lipid signaling. It has been shown that COX-2 expression is elevated in colon of Nrf2^{-/-} mice. Renal COX-1 and COX-2 expression are reduced in Keap1 ^{f/f} mice and increased in Nrf2^{-/-} mice and that the reduction in COX expression correlates with diminished production of the vasodilator PGI2.

Cyclooxygenase inhibitors have been used as a last-resort therapeutic for Li-NDI patients (Lam & Kjellstrand, Ren Fail 19, 183-8. (1997); Tran-Van. et al., Presse Med. 34, 1137-40 (2005)), despite the well-documented risk of acute kidney injury associated with this class of drugs (Ungprasert et al., Eur. J. Intern. Med. 26, 285-291 (2015)). Mice lacking the microsomal prostaglandin E synthase-1 (mPGES-1) were also found to be resistant to Li-NDI. However, in contrast to the Keap1^{f/f} mice used in the studies presented herein, these animals displayed normal AQP2 expression and maintained normal urine concentrating ability, suggesting a different physiologic mechanism underlying the protection. Without being bound by theory, Nrf2 may increase effectiveness of tubuloglomerular feedback via alteration of distal sodium reabsorption and modulation of vasodilator biosynthesis. On histologic examination, focal nephron atrophy and interstitial fibrosis are universally found in Li-associated nephropathy (Gitlin, Drug Saf 20, 231-43 (1999)). Preclinical studies have demonstrated that pharmacologic or genetic activation of Nrf2 can protect against acute and chronic kidney diseases by protecting cells from oxidative injury and reducing fibrotic remodeling. Rodent models of CKD have implicated Nrf2 deficiency as an important component of disease etiology (Kim & Vaziri, Am. J. Physiol. Renal Physiol. 298, F662-71 (2010); Kim et al., J. Pharmacol. Exp. Ther. 337, 583-590 (2011); Aminzadeh et al., Nephrol. Dial. Transplant. 28, 2038-2045 (2013)). Mice with constitutive hyperactivation of Nrf2 activity induced by genetic hypomorphism of Keap1 are protected against obstructive and ischemic kidney injury (Tan et al., Sci. Rep. 6, 36185 (2016)). Pharmacologic activators of Nrf2 are currently undergoing Phase II and III clinical trials for treatment of CKD (CXA-10 and CDDO-Me respectively).

The results presented herein show that pharmacologically activating Nrf2, such as with CDDO-Me, protected mice against acute development of Li-NDI. While several therapeutic options exist for treatment of established Li-NDI, Nrf2 activators may offer a unique advantage through primary prevention of renal injury. Unlike thiazides, amiloride, or acetazolamide, Nrf2 activation did not induce diuresis or otherwise affect volume homeostasis. A major complication with existing diuretic-based therapies is that volume depletion by these agents complicates the dosing of Li and other pharmaceuticals. As such, their use becomes difficult in the setting of polypharmacy and they are not ideal as a prophylactic medication. Nrf2 inducers can be administered as chronic prophylaxis in conjunction with Li, and may be administered safely prior to development of polyuria. In fact, the beneficial effects of Nrf2 activators in diverse models of renal fibrosis can provide bimodal protection in NDI: protection against development of acute NDI, and protection against the long-term sequela of fibrotic remodeling and CKD associated with chronic Li therapy.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method for treating or preventing nephrogenic diabetes insipidus (NDI) in a subject, comprising:
administering to the subject a therapeutically effective amount of a Nuclear factor-erythroid 2-related factor 2 (Nrf2) inducer,
thereby treating or preventing the NDI in the subject.

2. The method of claim 1, further comprising
selecting the subject with the NDI.

3. The method of claim 1, wherein the subject is human.

4. The method of claim 1, wherein the NDI is congenital NDI.

5. The method of claim 1, wherein the NDI is acquired NDI.

6. The method of claim 5, wherein the acquired NDI is lithium-induced NDI, hypokalemic nephropathy, hypercalcemia, and post-obstructive uropathy.

7. The method of claim 6, wherein the NDI is lithium-induced NDI.

8. The method of claim 6, wherein the subject has bipolar disorder.

9. The method of claim 1, further comprising administering a diuretic and/or a non-steroidal anti-inflammatory agent to the subject.

10. The method of claim 1, wherein the method decreases polyuria, or prevents the development of polyuria.

11. The method of claim 1, wherein the Nrf2 inducer is a fumarate, a nitro fatty acid, a bardoxolone, or sulforaphane.

12. The method of claim 1, wherein the Nrf2 inducer is a fumarate acid ester or fumaric acid.

13. The method of claim 1, wherein the Nrf2 inducer is dimethyl fumarate, diroximel fumarate, tepilamide fumarate, or monomethyl fumarate.

14. The method of claim 1, wherein the Nrf2 inducer is omaveloxolone, bardoxolone methyl, or bardoxolone-imidazole.

15. The method of claim 1, wherein the Nrf2 inducer is 9-nitro-octadec-9-enoic acid, 10-nitro-octadec-9-enoic acid, 9-nitro-tetradec-9-enoic acid, 10-nitro-tetradec-9-enoic acid, 10-nitro-pentadec-10-enoic acid, 11-nitro-pentadec-10-enoic acid, 7-nitro-nonadec-7-enoic acid, 8-nitro-nonadec-7-enoic acid, 8-nitro-eicos-8-enoic acid, 9-nitro-eicos-8-enoic acid, 6-nitro-octadec-6-enoic acid, or 7-nitro-octadec-6-enoic acid.

16. The method of claim 1, wherein the Nrf2 inducer is sulforaphane-cyclodextrin complex.

17. The method of claim 1, wherein the Nrf2 inducer is a nitro fatty acid administered at a daily dose of 75 mg, 150 mg or 300 mg.

18. The method of claim 1, wherein the Nrf2 inducer is a compound of: 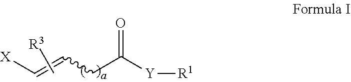
or a pharmaceutically acceptable salt, stereoisomer, and regioisomer thereof, wherein:
X is selected from H, 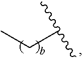
alkyl, substituted alkyl, alkenyl, nitroalkenyl, substituted alkenyl, and substituted nitroalkenyl;
Y is selected from NH, O, and S;
a is from 0-30;
b is from 0-30;
R¹ is selected from H, alkyl, substituted alkyl, haloalkyl, substituted haloalkyl, cycloalkyl, substituted cycloalkyl, aryl, substituted aryl, heteroaryl, substituted heteroaryl, -C(O)-R², gluconate, glycoside, glucuronide, tocopherols, and PEG groups; and
R² is selected from alkyl, substituted alkyl, haloalkyl, substituted haloalkyl, cycloalkyl, substituted cycloalkyl, aryl, substituted aryl, heteroaryl, and substituted heteroaryl; and
R³ is selected from H, OH, NO_{2,} C(O)H, C(O)-R², COOR², COON, CN, SO₃, SO₂R², SO₃H, Cl, Br, I, F, CF₃, CHF₂, and CH₂F.

19. The method of claim 17, wherein nitro fatty acid is administered as a single daily dose.

20. The method of claim 17, wherein nitro fatty acid is administered as a single dose twice a day.

21. A pharmaceutical composition comprising (i) a Nuclear factor-erythroid 2-related factor 2 (Nrf2) inducer and (ii) lithium or a lithium salt.
